(19) United States
(12) Patent Application Publication
(10) Pub. No.: US 2010/0114237 A1
(43) Pub. Date: May 6, 2010

(54) MOOD CIRCUIT MONITORING TO CONTROL THERAPY DELIVERY

(75) Inventors: Jonathon E. Giftakis, Maple Grove, MN (US); Mark T. Rise, Monticello, MN (US); David L. Carlson, Fridley, MN (US); Paul H. Stypulkowski, North Oaks, MN (US); Scott R. Stanslaski, Shoreview, MN (US); Randy M. Jensen, Hampton, MN (US); Timothy J. Denison, Minneapolis, MN (US)

Correspondence Address: SHUMAKER & SIEFFERT , P.A, 1625 RADIO DRIVE , SUITE 300, WOODBURY, MN, 55125, US

(73) Assignee: Medtronic, Inc.

(21) Appl. No.: 12/608,815

(22) Filed: Oct. 29, 2009

Related U.S. Application Data
(60) Provisional application No. 61/110,440, filed on Oct. 31, 2008.

(51) Int. Cl. A61N 1/00

(52) U.S. Cl. 607/45

(57) ABSTRACT

Brain signals may be monitored at different locations of a mood circuit in order to determine a mood state of the patient. A relationship (e.g., a ratio) between frequency band characteristics of the monitored brain signals may be indicative of a particular mood state. In some examples, therapy parameter values that define the therapy delivered to the patient may be selected to maintain a target relationship (e.g., a target ratio) between the frequency band characteristics of the brain signals monitored within the mood circuit. In addition, in some examples, therapy delivery to the patient may be controlled based on the frequency band characteristics of brain signals sensed at different portions of the mood circuit.

This application claims the benefit of U.S. Provisional Application No. 61/110,440, entitled, “MOOD CIRCUIT MONITORING TO CONTROL THERAPY DELIVERY,” and filed on Oct. 31, 2008, the entire content of which is incorporated herein by reference.

### TECHNICAL FIELD

The disclosure relates to medical devices and, more particularly, the configuration of therapy parameters.

### BACKGROUND

Implantable medical devices, such as electrical stimulators or therapeutic agent delivery devices, may be used in different therapeutic applications, such as deep brain stimulation (DBS), spinal cord stimulation (SCS), pelvic stimulation, gastric stimulation, peripheral nerve stimulation (PNS) or delivery of pharmaceutical agent, insulin, pain relieving agent or anti-inflammatory agent to a target tissue site within a patient. A medical device may be used to deliver therapy to a patient to treat a variety of symptoms or patient conditions such as chronic pain, tremor, Parkinson's disease, other types of movement disorders, seizure disorders (e.g., epilepsy), urinary or fecal incontinence, sexual dysfunction, obesity, mood disorders, gastroparesis or diabetes. In some cases, the electrical stimulation may be used for muscle stimulation, e.g., functional electrical stimulation (FES) to promote muscle movement or prevent atrophy. In some therapy systems, an implantable electrical stimulator delivers electrical therapy to a target tissue site within a patient with the aid of one or more electrodes, which may be deployed by medical leads. In addition to or instead of electrical stimulation therapy, a medical device may deliver a therapeutic agent to a target tissue site within a patient with the aid of one or more fluid delivery elements, such as a catheter or a therapeutic agent eluting patch.

During a programming session, which may occur during implant of the medical device, during a trial session, and/or during a follow-up session after the medical device is implanted in the patient, a clinician may generate one or more therapy programs that provide efficacious therapy to the patient, where each therapy program may define values for a set of therapy parameters. A medical device may deliver therapy to a patient according to one or more stored therapy programs. In the case of electrical stimulation, the therapy parameters may define characteristics of the electrical stimulation waveform to be delivered. Where electrical stimulation is delivered in the form of electrical pulses, for example, the parameters may include an electrode combination, an amplitude, which may be a current or voltage amplitude, a pulse width, and a pulse rate for the pulses. In the case of a therapeutic agent delivery device, the therapy parameters may include a dose (e.g., a bolus or a group of boluses) size, a frequency of bolus delivery, a concentration of a therapeutic agent in the bolus, a type of therapeutic agent to be delivered to the patient (if the medical device is configured to deliver more than one type of agent), a lock-out interval, and so forth.

### SUMMARY

In general, the disclosure is directed to devices, systems, and methods for delivering therapy to a patient to manage a psychiatric disorder (e.g., a mood disorder), which may be characterized by the presence of one or more patient mood states. The patient mood state may be a symptom of a psychiatric disorder with which the patient is afflicted. In some examples, brain signals may be monitored at different locations of a mood circuit of the brain in order to track a mood state of the patient. A relationship (e.g., a ratio) between frequency band characteristics of the monitored brain signals may be indicative of a particular mood state. In some examples, therapy parameter values that define the therapy delivered to the patient may be selected to maintain a target relationship (e.g., a ratio) between the frequency band characteristics of the brain signals monitored within the mood circuit. In addition, in some examples, a patient mood state may be detected based on the frequency band characteristics of brain signals sensed within the mood circuit. Therapy delivered to the patient may be controlled based on the detected mood state.

In one example, the disclosure is directed to a method comprising monitoring a first brain signal of a patient at a first location within the brain of the patient, monitoring a second brain signal at a second location within the brain, wherein the first and second locations are part of a common mood circuit of the brain, determining a mood state metric indicative of a relationship between a first frequency band characteristic of the first brain signal and a second frequency band characteristic of the second brain signal, and controlling delivery of therapy to the patient to control a psychiatric disorder based on the mood state metric.

In another example, the disclosure is directed to a medical system comprising a therapy module that delivers a psychiatric disorder therapy to a patient, a sensing module that monitors a first brain signal of a patient at a first location within the brain of the patient and monitors a second brain signal at a second location within the brain, wherein the first and second locations are part of a common mood circuit of the brain, and a processor. The processor determines a mood state metric indicative of a relationship between a first frequency band characteristic of the first brain signal and a second frequency band characteristic of the second brain signal, and controls the therapy module based on the mood state metric.

In another example, the disclosure is directed to a medical system comprising means for delivering therapy to a patient to control a psychiatric disorder, means for monitoring a first brain signal of the patient at a first location within the brain of the patient, means for monitoring a second brain signal at a second location within the brain, wherein the first and second locations are part of a common mood circuit of the brain, means for determining a mood state metric indicative of a relationship between a first frequency band characteristic of the first brain signal and a second frequency band characteristic of the second brain signal and means for controlling the means for delivering therapy based on the mood state metric.

In another example, the disclosure is directed to a computer-readable medium comprising instructions. The instructions cause a programmable processor to perform any part of the techniques described herein.

The details of one or more examples of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an example therapy system including an implantable medical device, a patient programmer, and a clinician programmer.

FIG. 2 is a schematic block diagram illustrating example components of the implantable medical device of FIG. 1.

FIG. 3 is a schematic block diagram illustrating example components of the patient programmer of FIG. 1.

FIG. 4 is a schematic block diagram illustrating example components of the clinician programmer of FIG. 1.

FIG. 5 is a flow diagram illustrating an example technique for controlling therapy delivery to the patient by monitoring brain signals at different locations of the same mood circuit of the brain of a patient.

FIG. 6 is flow diagram illustrating an example technique for determining a baseline parameter value associated with the mood state of a patient.

FIG. 7 is a flow diagram illustrating an example technique for adjusting a therapy program based on a determined patient mood state.

FIG. 8 is a flow diagram illustrating an example technique for controlling the delivery therapy to a patient based on a determined mood state.

FIG. 9 is a flow diagram illustrating an example technique for associating a mood state metric with a particular patient mood state.

FIG. 10 is a flow diagram illustrating an example technique for determining a target value for a mood state metric.

FIG. 11 is a schematic diagram illustrating different examples of a sensing module configured to sense one or more secondary indicators of patient mood state.

FIG. 12 is a flow diagram illustrating an example technique for comparing the mood state indicated by brain signals monitored at different locations of the same mood circuit to the mood state indicated by one or more secondary indicators.

FIGS. 13A and 13B are a flow diagram illustrating an example technique for programming a medical device based on brain activity within a mood circuit of a patient's brain.

### DETAILED DESCRIPTION

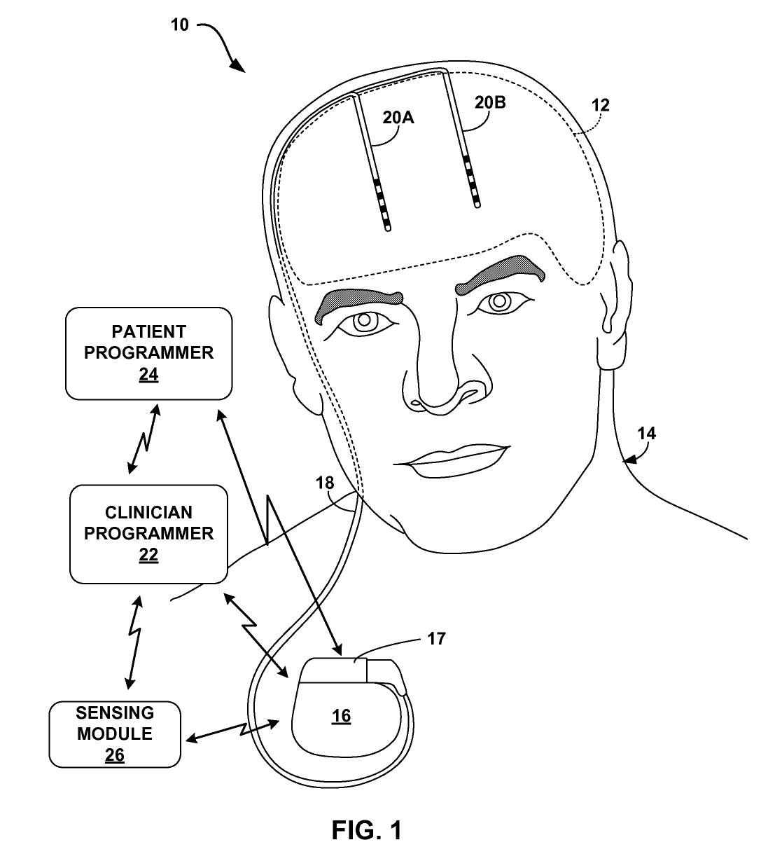

FIG. 1 is a conceptual diagram illustrating an example of therapy system 10 that is implanted to deliver therapy to brain 12 of patient 14 in order to help manage a patient condition, such as a psychiatric disorder. Examples of psychiatric disorders that therapy system 10 may be useful for managing include, but are not limited to, major depressive disorder (MDD), bipolar disorder, anxiety disorders, post traumatic stress disorder, dysthymic disorder, and obsessive-compulsive disorder (OCD). While patient 14 is generally referred to as a human patient, other mammalian or non-mammalian patients are also contemplated. Therapy system 10 includes implantable medical device (IMD) 16, connector block 17, lead extension 18, leads 20A and 20B, clinician programmer 22, patient programmer 24, and sensing module 26 (also referred to as “sensor 26”).

IMD 16 includes a therapy module that delivers electrical stimulation therapy to one or more regions of brain 12 via leads 20A and 20B (collectively referred to as “leads 20”). In the example shown in FIG. 1, therapy system 10 may be referred to as a deep brain stimulation (DBS) system because IMD 16 provides electrical stimulation therapy directly to tissue within brain 12, such as under the dura mater of brain 12. In addition to or instead of deep brain sites, the IMD 16 may deliver electrical stimulation to target tissue sites on a surface of brain 12, such as between the patient's cranium and the dura mater of brain 12 (e.g., the cortical surface of brain 12).

In the example shown in FIG. 1, IMD 16 may be implanted within a chest cavity of patient 14 or within a subcutaneous pocket below the clavical over the chest cavity of patient 14. In other examples, IMD 16 may be implanted within other regions of patient 14, such as a subcutaneous pocket in the abdomen of patient 14 or proximate the cranium of patient 14. Implanted lead extension 18 is mechanically and electrically connected to IMD 16 via connector block 17, which may include, for example, electrical contacts that electrically couple to respective electrical contacts on lead extension 18. The electrical contacts electrically couple the electrodes carried by leads 20A and 20B (collectively “leads 20”) to IMD 16. Lead extension 18 traverses from the implant site of IMD 16 within patient 14, along the neck of patient 14 and through the cranium of patient 14 to access brain 12.

Leads 20 are implanted within the right and left hemispheres, respectively, of brain 12 in order deliver electrical stimulation to one or more regions of brain 12, which may be selected based on many factors, such as the type of patient condition for which therapy system 10 is implemented to manage. In some examples, lead 20 may be implanted in the same hemisphere of brain 12. In addition, in some examples, electrodes of one or both leads 20 may be used to sense brain activity. Different neurological or psychiatric disorders may be associated with activity in one or more of regions of brain 12, which may differ between patients. For example, in the case of MDD, bipolar disorder or OCD, leads 20 may be implanted to deliver electrical stimulation to the anterior limb of the internal capsule of brain 12, or only the ventral portion of the anterior limb of the internal capsule and ventral portion of the striatum (also referred to as a VC/VS), the subgenual component of the cingulate cortex (e.g., cingulate area 25 (CG25)), anterior cingulate cortex Brodmann areas 32 and 24, various parts of the prefrontal cortex, including the dorsal lateral and medial pre-frontal cortex (PFC) (e.g., Brodmann areas 9 and 46), ventromedial prefrontal cortex (e.g., Brodmann area 10), the lateral and medial orbitofrontal cortex (e.g., Brodmann area 11), the medial or nucleus accumbens, thalamus, intralaminar thalamic nuclei, amygdala, hippocampus, the lateral hypothalamus, the Locus ceruleus, the dorsal raphe nucleus, ventral tegmentum, the substantia nigra, subthalamic nucleus, the inferior thalamic peduncle, the dorsal medial nucleus of the thalamus, or any combination thereof.

Although leads 20 are shown in FIG. 1 as being coupled to a common lead extension 18, in other examples, leads 20 may be coupled to IMD 16 via separate lead extensions or directly coupled to IMD 16. Leads 20 may deliver electrical stimulation to treat any number of neurological disorders or diseases in addition to psychiatric disorders, such as movement disorders or seizure disorders. Examples of movement disorders include a reduction in muscle control, motion impairment or other movement problems, such as rigidity, bradykinesia, rhythmic hyperkinesia, nonrhythmic hyperkinesia, dystonia, tremor, and akinesia. Movement disorders may be associated with patient disease states, such as Parkinson's disease or Huntington's disease. Examples of seizure disorders include epilepsy.

Leads 20 may be implanted within a desired location of brain 12 via any suitable technique, such as through respective burr holes in a skull of patient 14 or through a common burr hole in the cranium. Leads 20 may be placed at any location within brain 12 such that the electrodes of the leads are capable of providing electrical stimulation to targeted tissue during treatment. Electrical stimulation generated from the signal generator (not shown) within the therapy module of IMD 16 may help prevent the onset of events associated with the patient's psychiatric disorder or mitigate symptoms of the psychiatric disorder. For example, electrical stimulation therapy delivered by IMD 16 to a target tissue site within brain 12 may help prevent a manic event if patient 14 has a bipolar disorder or help patient 14 maintain a mood state between a manic state and a depressive state. The exact therapy parameter values of the stimulation therapy, such as the amplitude or magnitude of the stimulation signals, the duration of each signal, the waveform of the stimuli (e.g., rectangular, sinusoidal or ramped signals), the frequency of the signals, and the like, may be specific for the particular target stimulation site (e.g., the region of the brain) involved as well as the particular patient and patient condition.

In the case of stimulation pulses, the stimulation therapy may be characterized by selected pulse parameters, such as pulse amplitude, pulse rate, and pulse width. In addition, if different electrodes are available for delivery of stimulation, the therapy may be further characterized by different electrode combinations. Known techniques for determining the optimal stimulation parameters may be employed. In one example, electrodes of leads 20 are positioned to deliver stimulation therapy to an anterior limb of the internal capsule of brain 12 in order to manage symptoms of a MDD of patient 14, and stimulation therapy is delivered via a selected combination of the electrodes to the anterior limb of the internal capsule with electrical stimulation including a frequency of about 2 hertz (Hz) to about 2000 Hz, a voltage amplitude of about 0.5 volts (V) to about 50 V, and a pulse width of about 60 microseconds (μs) to about 4 milliseconds (ms). However, other examples may implement stimulation therapy including other stimulation parameters.

The electrodes of leads 20 are shown as ring electrodes. Ring electrodes may be relatively easy to program and are typically capable of delivering an electrical field to any tissue adjacent to leads 20. In other examples, the electrodes of leads 20 may have different configurations. For example, the electrodes of leads 20 may have a complex electrode array geometry that is capable of producing electrical fields having predefined shapes, e.g., that are selected based on the target tissue sites within brain 12 for the electrical stimulation. The complex electrode array geometry may include multiple electrodes (e.g., partial ring or segmented electrodes) around the perimeter of each lead 20, rather than a ring electrode. In this manner, electrical stimulation may be directed to a specific direction from leads 20 to enhance therapy efficacy and reduce possible adverse side effects from stimulating a large volume of tissue. In some examples, a housing of IMD 16 may include one or more stimulation and/or sensing electrodes. In alternative examples, leads 20 may have shapes other than elongated cylinders as shown in FIG. 1. For example, leads 20 may be paddle leads, spherical leads, bendable leads, or any other type of shape effective in treating patient 14.

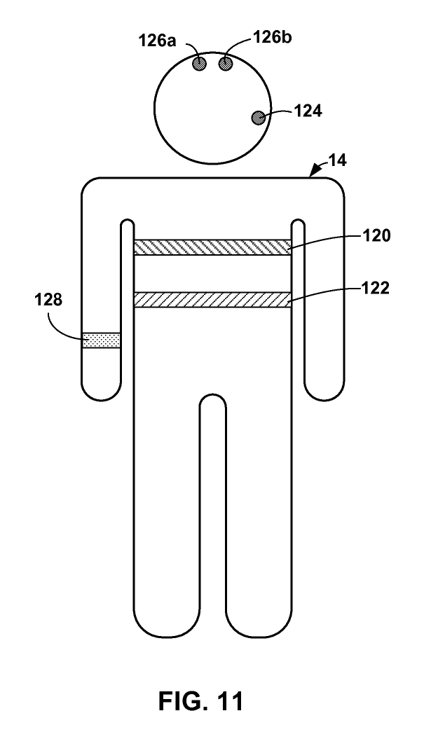

In some examples, leads 20 may include sensing electrodes positioned to detect electrical signals within one or more region of patient's brain 12. Alternatively, another set of sensing electrodes may monitor the electrical signal, such as those described with respect to FIG. 11. In general, the electrical signals within the patient's brain 12 may be interchangeably referred to herein as brain signals or bioelectrical brain signals. The brain signal may include a bioelectrical signal, such as an electroencephalogram (EEG) signal, an electrocorticogram (ECoG) signal, a local field potential (LFP) sensed from within one or more regions of patient's brain 12, and/or action potentials from single cells within the patient's brain. For example, the monitored brain signals may include an electroencephalogram (EEG) signal, which may be generated via one or more electrodes implanted and/or located external to patient 14. Electrodes implanted closer to the target region of brain 12 may help generate an electrical signal that provides more useful information than an EEG generated via a surface electrode array because of the proximity to brain 12. The EEG signal that is generated from an electrode array implanted within brain 12 may also be referred to as an electrocorticography (ECoG) signal.

As described in further detail below, in some examples, IMD 16 may monitor brain signals within different regions of brain 12 in order to control the delivery of psychiatric therapy to patient 14. Controlling therapy delivery may include, for example, initiating the delivery of electrical stimulation (or other therapy) to patient 14, adjusting one or more stimulation parameter values, adjusting the duty cycle of the delivery of a periodic electrical stimulation therapy or deactivating the delivery of electrical stimulation (or other therapy) to patient 14. In some examples, IMD 16 may monitor brain signals within different locations of a neurological mood circuit of brain 12 of patient 14 to detect a patient mood state. For example, a ratio of the energy levels (or power levels) within select frequency bands of the brain signals sensed at different parts of a mood circuit may indicate the patient mood state. In this way, the delivery of therapy to patient 14 via therapy system 10 may be controlled based on the power levels of selected frequency bands of the sensed brain signals.

The patient mood state may be a state in which one or more symptoms of a psychiatric disorder with which the patient is afflicted are apparent or otherwise perceived by patient 14. As examples, the patient mood state may include a depressive mood state, anxious mood state, obsessive-compulsive mood state, manic mood state, and the like. In addition, each of the aforementioned mood states may include multiple types of mood states that are depending on the severity of the patient's symptoms. For example, the depressive mood state may comprise a mild depressive mood state, moderate depressive mood state or a severe mood state. The severe depressive mood state may include more severe symptoms than the mild depressive mood state

A mood circuit of brain 12 may generally refer to regions of brain 12 that are connected to each another via neurological pathways, whereby activity within one region of brain 12 may affect activity within another region of brain 12 that is part of the same mood circuit. The regions of brain 12 that define a mood circuit may be substantially within one cerebral hemisphere of brain 12 or may span across both the left and right hemispheres of brain 12. The left and right cerebral hemispheres of brain 12 may be delineated along a midline (e.g., a line extending along a sagittal plane) of patient 14.

The portions of brain 12 that define a mood circuit that is related to the patient's psychiatric disorder may be identified based on, for example, imaging techniques, such as magnetoencephalography (MEG), positron emission tomography (PET), functional magnetic resonance imaging (fMRI), or diffusion MRI. For example, a clinician may image brain 12 when patient 14 is in a pathological psychiatric state (e.g., a depressive or manic mood state), and the clinician may image brain 12 after efficacious therapy is delivered to patient 14 to identify the regions of brain 12 that were affected by the efficacious therapy. Alternatively, or additionally, a clinician may apply brief electrical signals to a portion of brain 12 via one electrode on lead 20A, for example, and record the electrical signal from another electrode on lead 20A or, alternatively, on a second lead 20B. The recorded signal may have a specific pattern recognizable to the clinician if the stimulation electrode of lead 20A is physically located within the same mood circuit as the sense electrode of lead 20B. In some examples, the process may be repeated multiple times with the recorded signal linked in time with the applied stimulation. The evoked signal recorded may then be averaged over the multiple repetitions to enhance the signal strength relative to the background noise. The resulting averaged, evoked signal may be used to establish that the location at which the signal is sensed is part of the same mood circuit. Alternatively, the clinician may use established landmarks of brain anatomy developed through historical scientific investigation to, a priori, implant the therapy delivery lead in one portion of the mood circuit and implant the one or more sensing electrodes in other portions of the mood circuits.

The regions of the brain 12 that are part of a common mood circuit may be influenced at least in part by a particular mood state of patient 14. For example, when patient 14 is in a certain mood state, the activity of brain 12 at regions in a mood circuit may exhibit certain characteristics, such that the activity in one region of the mood circuit may exhibit behavior that is directly related to the behavior in another part of the mood circuit. The behavior of a region of brain 12 may be characterized by a frequency domain characteristic of a brain signal sensed within the region. An example of a frequency domain characteristic may include power level (or energy level) within a particular frequency band. The power level may be determined based on, for example, a spectral analysis of a bioelectrical brain signal. The spectral analysis may indicate the distribution over frequency of the power contained in a signal, based on a finite set of data.

In some examples, the frequency domain characteristic may comprise a relative power level in a particular frequency band. Thus, while “power levels” within a selected frequency band of a sensed brain signal are generally referred to herein, the power level may be a relative power level. A relative power level may include a ratio of a power level in a selected frequency band of a sensed brain signal to the overall power of the sensed brain signal. The power level in the selected frequency band may be determined using any suitable technique. In some examples, a processor of IMD 16 may average the power level of the selected frequency band of a sensed brain signal over a predetermined time period, such as about ten seconds to about two minutes, although other time ranges are also contemplated. In other examples, the selected frequency band power level may be a median power level over a predetermined range of time, such as about ten seconds to about two minutes. The activity within the selected frequency band of a brain signal, as well as other frequency bands of interest, may fluctuate over time. Thus, the power level in the selected frequency band at one instant in time may not provide an accurate and precise indication of the energy of the brain signal in the selected frequency band. Averaging or otherwise monitoring the power level in the selected frequency band over time may help capture a range of power levels, and, therefore, a better indication of the patient's pathological state in the particular brain region sensed by IMD 16.

The overall power of a sensed bioelectrical brain signal may be determined using any suitable technique. In one example, a processor of IMD 16 (or another device, such as a programmer 22, 24) may determine an overall power level of a sensed bioelectrical brain signal based on the total power level of a swept spectrum of the brain signal. To generate the swept spectrum, the processor may control sensing module 26 to tune to consecutive frequency bands over time, and the processor may assemble a pseudo-spectrogram of the sensed bioelectrical brain signal based on the power level in each of the extracted frequency bands. The pseudo-spectrogram may be indicative of the energy of the frequency content of the bioelectrical brain signal within a particular window of time.

In one accordance with example technique, the processor may determine an overall power level of a sensed bioelectrical brain signal based on time domain data. For example, the processor may determine the relative power in the selected frequency band by determining a ratio of the power in the selected frequency band to a voltage amplitude of the signal. The voltage amplitude may be a mean or median voltage amplitude of the brain signal over a predetermined range of time, such as about ten seconds to about two minutes, although other time ranges are also contemplated. The voltage amplitudes of the brain signals may be calibration coefficients that help minimize variability between the power levels of the bioelectrical brain signals in a particular frequency band that is attributable to differences in the overall signal power level.

As an example of the relationship between different portions of a mood circuit, when patient 14 is in a certain mood state, the power level of a brain signal sensed at one location of the mood circuit may increase while the power level of a brain signal sensed at a different location of the mood circuit may decrease. As another example, when patient 14 is in a certain mood state, the power level of a brain signal sensed at one location of the mood circuit may decrease or increase while the power level of a brain signal sensed at a different location of the mood circuit may also decrease or increase, respectively. As another example, when patient 14 is in a certain mood state, the power level of a brain signal sensed at one locations of the mood circuit may remain substantially constant while the power level of a brain signal sensed at a different location of the mood circuit may decrease or increase.

The power level of a brain signal in a selected frequency band may be determined using any suitable technique. In some examples, a processor of IMD 16 may average the power level of the selected frequency band of a sensed brain signal over a predetermined time period, such as about ten seconds to about two minutes, although other time ranges are also contemplated, e.g., about two minutes to about an hour. In other examples, the power level may be a median power level over a predetermined range of time, such as, e.g., about ten seconds to about two minutes or about two minutes to about an hour. The activity within the selected frequency band of a brain signal, as well as other frequency bands of interest, may fluctuate over time, e.g., due to the patient's normal brain activity or because of noise from external sources. Thus, the power level in the selected frequency band at one instant in time may not provide an accurate and precise indication of the energy of bioelectrical brain signal in the selected frequency band. Averaging or otherwise monitoring the power level in the selected frequency band over time may help capture a range of power levels, and, therefore, a better indication of the patient's pathological state in the particular brain region of the mood circuit.

In some examples, IMD 16 may track the power level of a brain signal in a particular frequency band by storing periodic power level determinations in a median filter. For example, the median power level may only be calculated when the median filter is full (block median). However a median filter that provides a rolling mean may also be used. In a rolling mean, the median power level is periodically calculated for the power level values stored in the median filter, regardless of whether the median filter is full.

If the mood state of patient 14 changes, the brain activity at the regions of brain 12 within the mood circuit associated with the mood state may be altered. As a result, the change in the brain activity within the mood circuit may be detected by detecting a change in one region of the mood circuit or detecting a change in multiple regions of the mood circuit relative to each another. Again, the changes may be detected based on the frequency domain characteristics of brain signals sensed within the one or more regions of the mood circuit.

In some cases, the power level within specific frequency bands of first and second brain signals sensed at different locations of brain 12 along a mood circuit may change relative to one another based on the mood state of the patient. For example, a majority of power of a first brain signal sensed within brain 12 at first location of the mood circuit may be within an alpha frequency band (e.g., approximately 5 Hertz (Hz) to approximately 13 Hz) and a majority of power of a second brain signal sensed within brain 12 at a second location of the mood circuit may be within a beta frequency band (approximately 13 Hz to approximately 30 Hz) when patient 14 is in a first mood state. In some examples, a brain signal may be sensed at the same location of the brain 12, and the power within two or more frequency bands of the signal at the brain location may be analyzed as described herein, e.g., to monitor patient mood state.

However, when the mood state of patient 14 changes, e.g., from the first mood state to a second mood state, the majority of power of the first brain signal sensed at the first location may shift from the alpha frequency band to the beta frequency band and the majority of the power of the second brain signal sensed at the second location may remain unchanged, e.g., in the beta frequency band. Accordingly, such a relationship of the frequency characteristics of the first and second brain signals sensed at different parts of a mood circuit may be identified (e.g., by a clinician) as being a biomarker for a certain patient mood state. IMD 16 may then use this known relationship between the frequency characteristics of the first and second brain signals sensed at different locations of a mood circuit to identify when patient 14 is in the associated mood state, which may then be used to control therapy delivery to patient 14.

In some examples, IMD 16 may be configured to monitor one or more frequency band characteristics of brain signals sensed at two or more locations of brain 12 that are a part of a common mood circuit. IMD 16 may monitor the frequency band characteristics and detect a particular mood state when the frequency band characteristics are in a predetermined relationship relative to each other. For example, if the frequency band characteristics comprise power levels within a particular frequency band of the sensed brain signals, the predetermined relationship that indicates the particular mood state may be a ratio of the power levels within a selected frequency band of a first brain signal and a second brain signal. Different patient mood states may be associated with different ratios of power levels within a selected frequency band of a first brain signal and a second brain signal. The selected frequency band may differ depending on the patient mood state. For example, IMD 16 or sensing module 26 may tune to different frequency bands depending upon the psychiatric disorder of patient 14. The mood states and associated ratios or other predetermined relationships may be stored within a memory of IMD 16.

If IMD 16 detects that two or more brain signals within a mood circuit exhibit a predetermined relationship associated with the patient mood state, IMD 16 may determine that patient 14 is in the mood state corresponding to the predetermined relationship. Based on this determination, IMD 16 may control the therapy delivery to patient 14 to effectively manage a mood disorder of patient 14. For example, IMD 16 may modify one or more stimulation parameter values for the electrical stimulation delivered by IMD 16 based on the detected mood state. IMD 16 may modify the one or more stimulation parameter values by adjusting the stimulation parameter value or switching therapy programs or program groups. As described in further detail below, a therapy program may define a set of stimulation parameter values for the stimulation therapy generated and delivered by IMD 16 and a program group may comprise two or more therapy programs.

A conceptual illustration of sensing module 26 is shown in FIG. 1. Sensing module 26 may be external to patient 14, may be implanted within patient 14 or may include portions both implanted and external to patient 14. In some examples, sensing module 26 may be incorporated in a common housing with IMD 16, may be electrically connected to electrodes on an outer housing of IMD 16 or on leads 20 or separate leads extending from IMD 16.

Sensing module 26 may monitor one or more physiological signals of patient 14. In some examples, the physiological signals may include the brain signals, which may be sensed at two or more locations along the same mood circuit, as described above. Sensing module 26 of therapy system 10 may monitor the one or more brain signals within brain 12 instead of or in addition to IMD 16. In some examples, sensing module 26 may monitor (or sense) brain signals at two or more locations along a mood circuit by monitoring an EEG signal sensed by two or more external electrodes, e.g., scalp electrodes. In other examples, sensing module 26 may monitor brain signals at two or more locations along a mood circuit by monitoring an ECoG signal sensed by two more electrodes implanted within patient 14, e.g. electrodes implanted within brain 12 of patient. In any case, the electrodes may be positioned relative to brain 12 of patient 14 in a manner that allows system 10 to monitor brain signals at two or more locations along the same mood circuit to control the delivery of therapy to patient 14 by IMD 16.

In some examples, sensing module 26 may include circuitry to tune to and extract a power level of a particular frequency band of a sensed brain signal. Thus, the power level of a particular frequency band of a sensed brain signal may be extracted prior to digitization of the signal by processor 34. By tuning to and extracting the power level of a particular frequency band before the signal is digitized, it may be possible to run frequency domain analysis algorithms at a relatively slower rate compared to systems that do not include a circuit to extract a power level of a particular frequency band of a sensed brain signal prior to digitization of the signal. In some examples, sensing module 26 may include more than one channel to monitor simultaneous activity in different frequency bands, i.e., to extract the power level of more than one frequency band of a sensed brain signal. These frequency bands may include an alpha frequency band (e.g., approximately 5 Hz to approximately 13 Hz), beta frequency band, or other frequency bands.

Changes to the patient's mood state may not be sudden and may change relatively slowly over time, as compared to, for example, the onset of a seizure. Accordingly, brain signals sensed within two or more parts of brain 12 of patient 14 may be sampled at a relatively slow rate in order to monitor the patient's mood state, which may be used to control IMD 16. In some examples, a sampling rate of brain signals of about 0.5 Hertz or slower may be used, although other sampling frequencies are also contemplated. In some examples, sensing module 26 may apply a low pass filter to a sensed brain signal in order to smooth the brain signal.

In some examples, sensing module 26 may include an architecture that merges chopper-stabilization with heterodyne signal processing to support a low-noise amplifier. In some examples, sensing module 26 may include a frequency selective signal monitor that includes a chopper-stabilized superheterodyne instrumentation amplifier and a signal analysis unit. Example amplifiers that may be included in the frequency selective signal monitor are described in further detail in commonly-assigned U.S. Patent Publication No. 2009/0082691 to Denison et al., entitled, “FREQUENCY SELECTIVE MONITORING OF PHYSIOLOGICAL SIGNALS” and filed on Sep. 25, 2008. U.S. Patent Publication No. 2009/0082691 to Denison et al. is incorporated herein by reference in its entirety.

As described in U.S. Patent Publication No. 2009/0082691 to Denison et al., frequency selective signal monitor may utilize a heterodyning, chopper-stabilized amplifier architecture to convert a selected frequency band of a physiological signal to a baseband for analysis. The physiological signal may include a bioelectrical brain signal, which may be analyzed in one or more selected frequency bands to select a stimulation electrode combination in accordance with the techniques described herein. The frequency selective signal monitor may provide a physiological signal monitoring device comprising a physiological sensing element that receives a physiological signal, an instrumentation amplifier comprising a modulator that modulates the signal at a first frequency, an amplifier that amplifies the modulated signal, and a demodulator that demodulates the amplified signal at a second frequency different from the first frequency. A signal analysis unit may analyze a characteristic of the signal in the selected frequency band. The second frequency may be selected such that the demodulator substantially centers a selected frequency band of the signal at a baseband.

In some examples, sensing module 26 may sense brain signals substantially at the same time that IMD 16 delivers therapy to patient 14. In other examples, sensing module 26 may sense brain signals and IMD 16 may deliver therapy at different times.

As described in further detail with reference to FIG. 11, in some examples, sensing module 26 may be configured to monitor one or more physiological parameters that provide a secondary indicator of patient mood state instead of or in addition to sensing brain signals. The physiological parameters may include physiological signals in addition to or other than brain signals. For example, the physiological parameters may include, but are not limited to, brain activity, heart rate, respiratory rate, electrodermal activity (e.g., skin conductance level or galvanic skin response), muscle activity (e.g., via electromyogram), thermal sensing (e.g. to detect facial flushing), or cardiac Q-T interval.

Brain activity may be indicated by, for example, monitoring electrical signals of the brain, such as EEG or ECoG signals. The heart rate and respiratory rate may be determined by measuring the heart rate and respiratory rate at any suitable place on the patient's body, and need not be directly measured from the heart or lungs. The electrodermal and thermal activity of patient 14 may be measured at the patient's face or any other suitable place on the patient's body, such as on the patient's hands (e.g., the palms), arms, legs, torso, neck, and the like. Thermal activity may indicate, for example, the temperature of the patient's skin due to skin flushing or an increase in blood flow. Monitoring the patient's muscle activity may detect changes to the patient's demeanor, such as changes to the patient's facial features (e.g., by detect facial contraction), tensing of the patient's neck and should muscles, clenching of the patient's hands, and the like.

A cardiac Q-T interval is a measure of the time between the start of the Q wave of the heart's electrical cycle and the end of the T wave, and is typically dependent upon the heart rate. Respiratory rate, heart rate, electrodermal activity, facial flushing, and cardiac Q-T interval signals may each be indicative of the patient's anxiety level. For example, a relatively high respiratory rate, heart rate, electrodermal activity, facial flushing, and Q-T interval may be indicative of a relatively high anxiety level of patient 14.

Sensing module 26 may include electrodes positioned on the patient's face in order to detect the electrical potential generated by the patient's facial muscle cells when the patient's face contracts. That is, in some embodiments, sensing module 26 may include one or more electrodes positioned to detect electromyography (EMG) signals, which may indicate changes to the patient's facial expressions. Certain EMG signals may be associated with particular facial expressions, e.g., during a learning process. In some embodiments, sensing module 26 may include one or more thermal sensing electrodes positioned on the patient's face in order to detect facial flushing, and/or one or more sensing electrodes to detect electrodermal activity, which may indicate changes in conductivity of the patient's skin (e.g., attributable to perspiration). In addition to or instead of the EMG or thermal sensing electrodes, sensing module 26 may include a respiration belt or an electrocardiogram (ECG) belt, as described below with reference to FIG. 11.

IMD 16 and sensing module 26 may communicate with each other, such that IMD 16 may receive an indication of the sensed physiological signals indicative of patient mood state. IMD 16 may determine whether the mood state determination based on the brain signals monitored at two or more locations along the same mood circuit and the mood state determination based on the secondary indicators are consistent. IMD 16 may control therapy delivery to patient 14 based on whether the mood state determinations are consistent.

IMD 16 includes a therapy module that generates the electrical stimulation delivered to patient 14 via electrodes of leads 20. In the example shown in FIG. 1, IMD 16 generates the electrical stimulation according to one or more therapy parameters, which may be arranged in a therapy program (or a parameter set). In particular, a signal generator (not shown) within IMD 16 produces the stimulation in the manner defined by the therapy program or group of programs selected by the clinician and/or patient 14. The signal generator may be configured to produce electrical pulses to treat patient 14. In other examples, the signal generator of IMD 16 may be configured to generate a continuous wave signal, e.g., a sine wave or triangle wave. In either case, IMD 16 generates the electrical stimulation therapy for DBS according to therapy parameter values defined by a particular therapy program.

As indicated above, a therapy program defines values for a number of parameters that define the stimulation. The therapy parameters may include, for example, voltage or current amplitudes, frequency, duty cycle, and electrode combinations, and, in the case of stimulation pulses, pulse widths, pulse rates, and the like. An electrode combination may indicate the subset of electrodes of leads 20 that are selected to deliver the electrical stimulation to brain 12, and, in some cases, the polarity of the selected electrodes. IMD 16 may store a plurality of programs. In some cases, the one or more stimulation programs are organized into groups, and IMD 16 may deliver stimulation to patient 14 according to a program group. During a trial stage in which IMD 16 is evaluated to determine whether IMD 16 provides efficacious therapy to patient 14, the stored programs may be tested and evaluated for efficacy.

IMD 16 may include a memory to store one or more therapy programs (e.g., arranged in groups), and instructions defining the extent to which patient 14 may adjust therapy parameters, switch between programs, or undertake other therapy adjustments. Patient 14 may generate additional programs for use by IMD 16 via patient programmer 24 at any time during therapy or as designated by the clinician.

Generally, an outer housing of IMD 16 may be constructed of a biocompatible material that resists corrosion and degradation from bodily fluids. The housing may be hermetically sealed to help protect internal components of IMD 16 (e.g., a processor or signal generator) from external environmental contaminants. IMD 16 may be implanted within a subcutaneous pocket close to the stimulation site. Although IMD 16 is implanted near a chest cavity of patient 14 in the example shown in FIG. 1, in other examples, IMD 16 may be implanted within cranium. In addition, while IMD 16 is shown as implanted within patient 14 in FIG. 1, in other examples, IMD 16 may be located external to patient 14. For example, IMD 16 may be a trial stimulator electrically coupled to leads 20 via a percutaneous lead during a trial period. If the trial stimulator indicates therapy system 10 provides effective treatment to patient 14, the clinician may implant a chronic stimulator within patient 14 for long term treatment.

Clinician programmer 22 may be a computing device including, for example, a personal digital assistant (PDA), a laptop computer, a desktop PC, a workstation, and the like that permits a clinician to program electrical stimulation therapy for patient 14, e.g., using input keys and a display. For example, using clinician programmer 22, the clinician may specify therapy programs that include one or more therapy parameters and/or organize the therapy programs into therapy program groups (i.e., groups including one or more therapy parameters) for use in delivery of DBS. Clinician programmer 22 supports telemetry (e.g., radio frequency (RF) telemetry) with IMD 16 to download stimulation parameters and, optionally, upload operational or physiological data stored by IMD 16. In this manner, the clinician may periodically interrogate IMD 16 to evaluate efficacy and, if necessary, modify the stimulation parameters. Clinician programmer 22 may also be used to download information relating to the patient's psychiatric disorder, such as mood states detected by IMD 16 based on the brain signals within different parts of a mood circuit and/or the secondary indicators of mood state, and the dates and times at which the mood states were detected.

Like clinician programmer 22, patient programmer 24 may be a handheld computing device. Patient programmer 24 may also include a display and input keys to allow patient 14 to interact with patient programmer 24 and IMD 16. In this manner, patient programmer 24 provides patient 14 with an interface for limited control of electrical stimulation therapy provided by IMD 16. For example, patient 14 may use patient programmer 24 to start, stop or adjust electrical stimulation therapy. In particular, patient programmer 24 may permit patient 14 to adjust stimulation parameters such as duration, amplitude, pulse width and pulse rate within an adjustment range specified by the clinician via clinician programmer 22, select from a library of stored stimulation therapy programs, or reset the current therapy cycle.

Patient programmer 24 includes input mechanisms to allow patient 14 to enter information related to a patient event or information in response to the delivery of therapy according to a particular therapy program. For example, any of the above-listed input mechanisms may be used to enter information including, but not limited to, information characterizing the patient mood state at different times, e.g., in order to assess whether IMD 16 is providing sufficient therapy to manage the patient's psychiatric disorder. The information entered by patient 14 may be associated with the specific therapy program. This may help a clinician evaluate the efficacy of a therapy program.

Clinician programmer 22 may be used to program and/or interrogate IMD 16 and patient programmer 24, as described in further detail below. IMD 16, clinician programmer 22, and patient programmer 24 may communicate via cables or a wireless communication, as shown in FIG. 1. Clinician programmer 22 and patient programmer 24 may, for example, communicate via wireless communication with IMD 16 using RF telemetry techniques known in the art. Clinician programmer 22 and patient programmer 24 also may communicate with each other using any of a variety of local wireless communication techniques, such as RF communication according to the 802.11 or Bluetooth specification sets, infrared communication, e.g., according to the IrDA standard, or other standard or proprietary telemetry protocols.

Although IMD 16 configured to deliver electrical stimulation is illustrated in the example shown in FIG. 1, in other examples, therapy system 10 may include a medical device configured to deliver a therapeutic agent in addition to or instead of electrical stimulation. The therapeutic agent may be used to provide therapy to patient 14 to manage a psychiatric disorder of patient 14, and may be delivered to the patient's brain 12, blood stream or tissue. In some examples, the medical device that delivers the therapeutic agent is implanted within patient 14, while in other examples, the medical device is external to patient 14. For example, the medical device may be an implanted or external drug pump that delivers a therapeutic agent to a target tissue site within patient 14 with the aid of one or more catheters. As another example, the medical device may be an external patch that is worn on a skin surface of patient 14, where the patch elutes a therapeutic agent, which is then absorbed by the patient's skin. Other types of therapeutic agent delivery systems are contemplated.

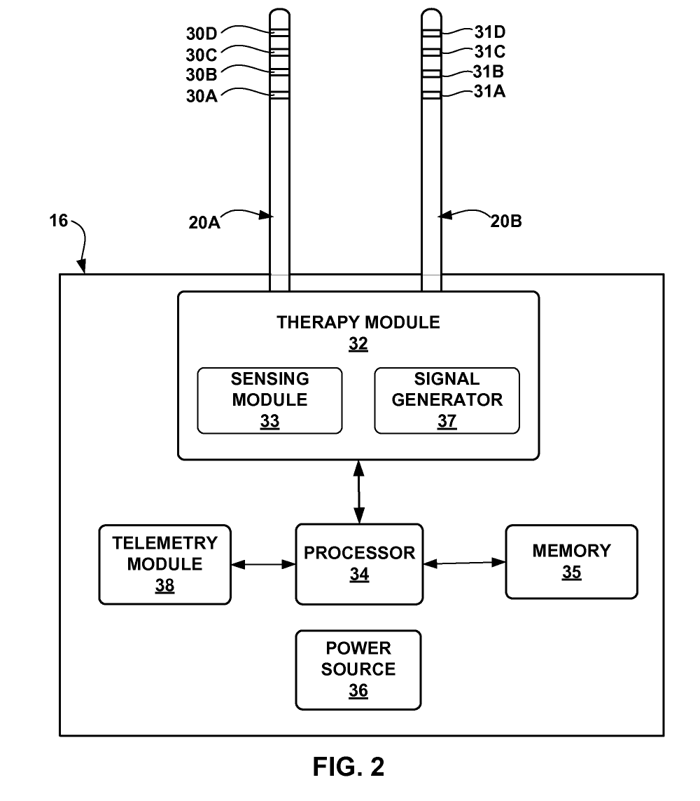

FIG. 2 is a functional block diagram illustrating components of an example of IMD 16 in greater detail. IMD 16 is coupled to leads 20A and 20B, which include electrodes 30A-D and 31A-D, respectively. Although IMD 16 is coupled directly to leads 20, in other examples, IMD 16 may be coupled to leads 20 indirectly, e.g., via lead extension 18 (FIG. 1). IMD 16 includes therapy module 32, processor 34, memory 35, power source 36, and telemetry module 38. In the example shown in FIG. 2, therapy module 32 includes sensing module 33 and signal generator 37. Sensing module 33 may be similar to sensing module 26 (FIG. 1), and may sense bioelectrical brain signals, as well as other physiological parameters of patient 14, such as the parameters described with respect to secondary indicators of patient mood state.

Signal generator 37 of therapy module 32 may deliver electrical stimulation therapy to brain 12 of patient 14 via a selected subset of electrodes 30A-D of lead 20A and electrodes 31A-D of lead 20B (collectively “electrodes 30 and 31”). In addition, sensing module 33 may sense bioelectrical brain signals of patient 14 via selected subset of electrodes 30, 31. Signal generator 37 may generate and deliver electrical signals (e.g., pulses or substantially continuous-time signals, such as sinusoidal signals) to a target tissue site within patient 14 via at least some of electrodes 30, 31 under the control of processor 34. In some examples, the stimulation energy generated by signal generator 37 may be delivered to selected electrodes 30, 31 via a switching module and conductors carried by leads 16, as controlled by processor 34. Similarly, in some examples, a select subset of electrodes 30, 31 may be electrically connected to sensing module 33 with the aid of a switching module. The switching module may include a switch array, switch matrix, multiplexer, or any other type of switching device suitable to selectively couple stimulation energy to selected electrodes. However, in some examples, IMD 16 may not include a switching module.

In the example shown in FIG. 2, implantable medical leads 20 are substantially cylindrical, such that electrodes 30, 31 are positioned on a rounded outer surface of leads 20. As previously described, in other examples, leads 20 may be, at least in part, paddle-shaped (i.e., a “paddle” lead). In some examples, electrodes 30, 31 may be ring electrodes. In other examples, electrodes 30, 31 may be segmented or partial ring electrodes, each of which extends along an arc less than 360 degrees (e.g., 90-120 degrees) around the outer perimeter of the respective lead 20. The use of segmented or partial ring electrodes 30, 31 may also reduce the overall power delivered to electrodes 30, 31 by IMD 16 because of the ability to more efficiently deliver stimulation to a target stimulation site by eliminating or minimizing the delivery of stimulation to unwanted or unnecessary regions within patient 16.

The configuration, type, and number of electrodes 30, 31 illustrated in FIG. 2 are merely exemplary. For example, IMD 16 may be coupled to one lead with eight electrodes on the lead or three or more leads with the aid of bifurcated lead extensions. Electrodes 30, 31 are electrically coupled to a therapy module 32 of IMD 16 via conductors within the respective leads 20A, 20B. Each of electrodes 30, 31 may be coupled to separate conductors so that electrodes 30, 31 may be individually selected, or in some examples, two or more electrodes 30 and/or two or more electrodes 31 may be coupled to a common conductor.

Processor 34 may include any one or more of a microprocessor, a controller, a digital signal processor (DSP), an application specific integrated circuit (ASIC), a field programmable gate array (FPGA), discrete logic circuitry, or the like, and the functions attributed to processor 34 may be embodied as software, firmware, hardware or any combination thereof. Processor 34 controls signal generator 37 to deliver electrical stimulation therapy according to selected therapy parameters defined by therapy programs. Specifically, processor 34 may control signal generator 37 to generate and deliver electrical signals with selected voltage or current amplitudes, pulse widths (if applicable), and rates specified by one or more therapy programs, which may be arranged into therapy program groups. In one example, processor 34 controls signal generator 37 to deliver stimulation therapy according to one therapy program group at a time. The therapy programs may be stored within memory 35. In another example, therapy programs are stored within at least one of clinician programmer 22 or patient programmer 24, which transmits the therapy programs to IMD 16 via telemetry module 38.

Processor 34 may also control signal generator 37 to deliver the electrical stimulation signals via selected subsets of electrodes 30, 31 with selected polarities. For example, electrodes 30, 31 may be combined in various bipolar or multi-polar combinations to deliver stimulation energy to selected sites, such as sites within brain 12. The above-mentioned switch matrix may be controlled by processor 34 to configure electrodes 30, 31 in accordance with a therapy program.

In examples in which IMD 16 monitors brain signals at one or more locations of a mood circuit of patient 14, processor 34 may control sensing module 33 of therapy module 32 to sense the brain signal at each location along the mood circuit. The sensed brain signals sensed by sensing module 33 may be stored within memory 35. Memory 35 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, and the like. Memory 35 may store program instructions that, when executed by processor 34, cause IMD 16 to perform the functions ascribed to IMD 16 herein. In some examples, memory 35 may also store the parameters for therapy programs or program groups and/or patient physiological data (such as sensed physiological signals) obtained by IMD 16 or another sensing device.

During a trial session, which may occur after implantation of IMD 16 or prior to implantation of IMD 16, a clinician may determine the therapy parameter values that provide efficacious therapy to patient 14. Processor 34 may control therapy module 32 based on information provided by clinician programmer 22, patient programmer 24 or another computing device. For example, the clinician may interact with clinician programmer 22 to select a particular therapy program and clinician programmer 22 may transmit a control signal to IMD 16, which is received by telemetry module 38 of IMD 16. The control signal may cause processor 34 to control signal generator 37 of therapy module 32 to deliver therapy based on the parameter values specific by the clinician-selected therapy program. As another example, clinician programmer 22, patient programmer 24 or another computing device may utilize a search algorithm that automatically selects therapy programs for trialing, i.e., testing on patient 14. When a therapy program is trialed, therapy is delivered to patient 14 according to the therapy program for a predetermined amount of time, which may be a few minutes to a few hours or days, in order to assess the efficacy of the therapy program in managing the patient's condition. The efficacy of the therapy program may be analyzed in terms of the therapeutic benefits to patient 14, as well as the existence of side effects, which may include the presence, severity, and duration of the side effects.

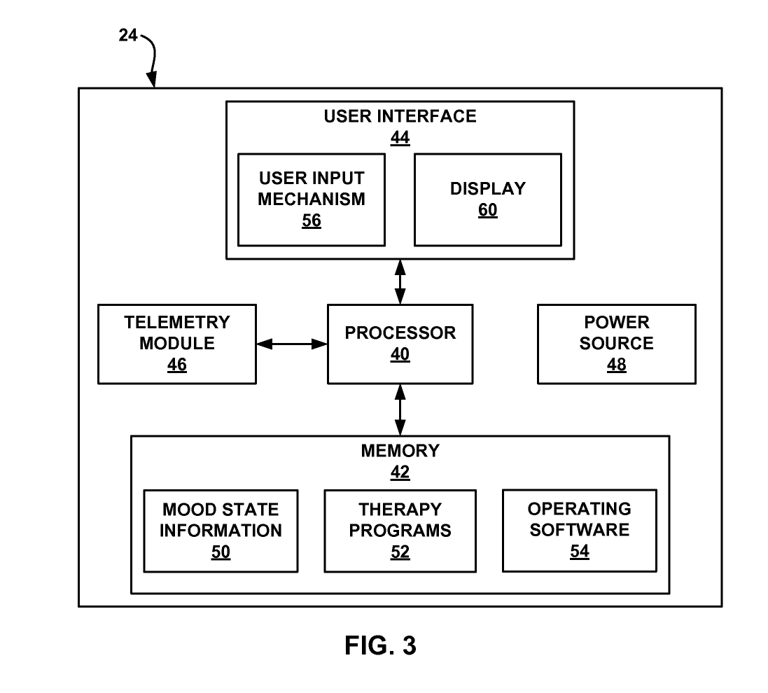

FIG. 3 is a functional block diagram illustrating components of an example patient programmer 24, which includes processor 40, memory 42, user interface 44, telemetry module 46, and power source 48. Processor 40 controls user interface 44 and telemetry module 46, and stores and retrieves information and instructions to and from memory 42. Patient programmer 24 may be a dedicated hardware device with dedicated software for programming of IMD 16. Alternatively, patient programmer 24 may be an off-the-shelf computing device running an application that enables programmer 24 to program IMD 16.

Patient 14 may use patient programmer 24 to select therapy programs (e.g., sets of stimulation parameters), generate new therapy programs, modify therapy programs through individual or global adjustments or transmit the new programs to a medical device, such as IMD 16 (FIGS. 1 and 2). Patient 14 may interact with patient programmer 24 via user interface 44, which includes user input mechanism 56 and display 60. In some examples, patient 14 may input information via user interface 44 relating to the therapeutic efficacy of a therapy program or a mood state during before, during and/or after therapy delivery by IMD 16.

User input mechanism 56 may include any suitable mechanism for receiving input from patient 14 or another user. In one example, user input mechanism includes an alphanumeric keypad. In another example, user input mechanism 56 includes a limited set of buttons that are not necessarily associated with alphanumeric indicators. For example, the limited set of buttons may include directional buttons that permit patient 14 to scroll up, down, or sideways through a display presented on display 60, select items shown on display 60, as well as enter information. The limited set of buttons may also include “increment/decrement” buttons in order to increase or decrease a stimulation frequency or amplitude of stimulation delivered by IMD 16.

User input mechanism 56 may include any one or more of push buttons, soft-keys that change in function depending upon the section of the user interface currently viewed by the user, voice activated commands, activated by physical interactions, magnetically triggered, activated upon password authentication push buttons, contacts defined by a touch screen, or any other suitable user interface. In some examples, buttons of user input mechanism 56 may be reprogrammable. That is, during the course of use of patient programmer 24, the buttons of user input mechanism 56 may be reprogrammed to provide different programming functionalities as the needs of patient 14 change or if the type of IMD 16 implanted within patient 14 changes. User input mechanism 56 may be reprogrammed, for example, by clinician programmer 22 (FIG. 1) or another computing device.

Display 60 may include a color or monochrome display screen, such as a liquid crystal display (LCD), light emitting diode (LED) display or any other suitable type of display. Patient programmer 24 may present information related to stimulation therapy provided by IMD 16, as well as other information, such as historical data regarding the patient's condition and past patient mood state information. Processor 46 may monitor activity from user input mechanism 56, and control display 60 and/or IMD 16 function accordingly. In some examples, display 60 may be a touch screen that enables the user to select options directly from the display. In such cases, user input mechanism 56 may be eliminated, although patient programmer 24 may include both a touch screen and user input mechanism 56. In some examples, user interface 44 may also include audio circuitry for providing audible instructions or sounds to patient 14 and/or receiving voice commands from patient 14.

User interface 44 may also include an LED or another indication (e.g., via display 60) that provides confirmation to patient 14 that an operation was carried out or that information input via user input mechanism 56 was received. For example, at certain times, user interface 44 may prompt patient 14 to provide feedback regarding the patient's mood state. Based on the received patient input, in some examples, IMD 16 may associate the indicated mood state with one or more characteristics (e.g., frequency band characteristics) of respective brain signals monitored at two or more locations along a brain circuit relative to one another, as described herein. For example, IMD 16 may associate the indicated mood state with a relationship between frequency band characteristics of brain signals sensed at different locations of a mood circuit at the time the mood state was indicated or prior to (e.g., within about one minute to about five minutes) the time in which patient 14 provided input indicating the mood state. After patient 14 provides feedback, user interface 44 may activate an LED to provide positive feedback to patient 14 regarding the successfully received information.

Processor 40 may comprise any combination of one or more processors including one or more microprocessors, DSPs, ASICs, FPGAs, or other equivalent integrated or discrete logic circuitry. Accordingly, processor 40 may include any suitable structure, whether in hardware, software, firmware, or any combination thereof, to perform the functions ascribed herein to processor 40. Memory 42 may include any volatile or nonvolatile memory, such as RAM, ROM, EEPROM or flash memory. Memory 42 may also include a removable memory portion that may be used to provide memory updates or increases in memory capacities. A removable memory may also allow patient data to be easily transferred to clinician programmer 22, or to be removed before patient programmer 24 is used by a different patient.

Memory 42 stores, among other things, mood state information 50, therapy programs 52, and operating software 54. Memory 42 may have any suitable architecture. For example, memory 42 may be partitioned to store mood state information 50, therapy programs 52, and operating software 54. Alternatively, mood state information 50, therapy programs 52, and operating software 54 may each include separate memories that are linked to processor 40.

Therapy programs 52 portion of memory 42 stores data relating to the therapy programs implemented by IMD 16 (FIG. 1). In some examples, the actual settings for the therapy programs, e.g., the stimulation amplitude, pulse rate, pulse frequency and pulse width data, are stored within therapy programs 52, and processor 40 may transmit the therapy parameter values to IMD 16. In other examples, an indication of each therapy program or group of therapy programs, e.g., a single value associated with each therapy program or group, may be stored within therapy programs 52, and the actual parameters may be stored within memory 35 (FIG. 2) of IMD 16. The “indication” for each therapy program or group may include, for example, alphanumeric indications (e.g., Therapy Program Group A, Therapy Program Group B, and so forth), or symbolic indications.

Operating software 54 may include instructions executable by processor 40 for operating user interface 44 and telemetry module 46, as well as for managing power source 48. Memory 42 may also store any therapy data retrieved from IMD 16 during the course of therapy. The clinician may use this therapy data to determine the progression of the patient's disease in order to predict or plan a future treatment.

Patient programmer 24 may communicate via wireless telemetry with IMD 16, such as using RF communication or proximal inductive interaction. This wireless communication is possible through the use of telemetry module 46. Accordingly, telemetry module 46 may be similar to the telemetry module contained within IMD 16. Telemetry module 46 may also be configured to communicate with clinician programmer 22 or another computing device via wireless communication techniques, or direct communication through a wired connection. Examples of local wireless communication techniques that may be employed to facilitate communication between patient programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with patient programmer 24 without needing to establish a secure wireless connection.

Power source 48 delivers operating power to the components of patient programmer 24. Power source 48 may include a battery and a power generation circuit to produce the operating power. In some examples, the battery may be rechargeable to allow extended operation. Recharging may be accomplished electrically coupling power source 48 to a cradle or plug that is connected to an alternating current (AC) outlet. In addition, recharging may be accomplished through proximal inductive interaction between an external charger and an inductive charging coil within patient programmer 24. In other examples, traditional batteries (e.g., nickel cadmium or lithium ion batteries) may be used. In addition, patient programmer 24 may be directly coupled to an alternating current outlet recharge power source 48, or to power patient programmer 24. Power source 48 may include circuitry to monitor power remaining within a battery. In this manner, user interface 44 may provide a current battery level indicator or low battery level indicator when the battery needs to be replaced or recharged. In some cases, power source 48 may be capable of estimating the remaining time of operation using the current battery.

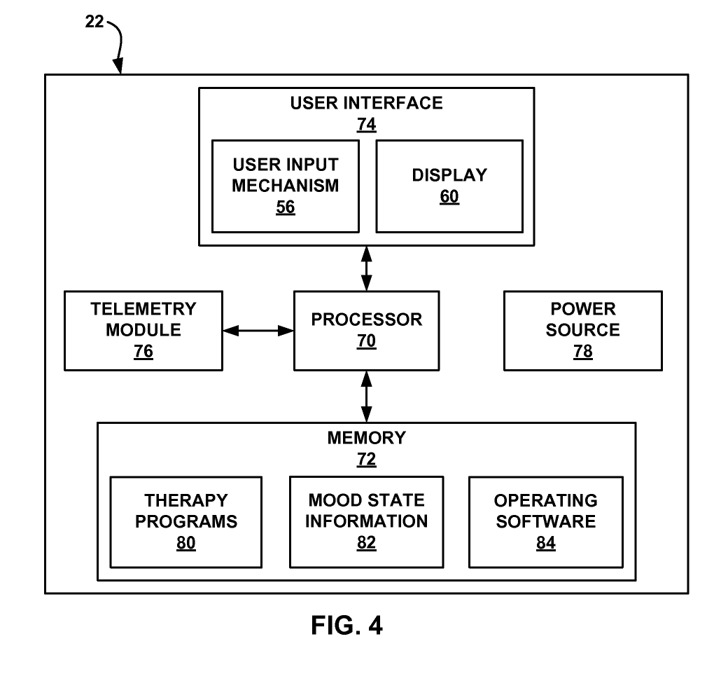

FIG. 4 is a functional block diagram illustrating components of clinician programmer 22, which may be similar to patient programmer 24. Clinician programmer 22 may include processor 70, memory 72 including therapy programs 80, mood state information 82, and operating software 84, user interface 74 including user input mechanism 56 and display 60, telemetry module 76, and power source 78.

The functions performed by each component may be similar to the functions described above with reference to the similar components of patient programmer 24. Additionally, clinician programmer 22 may include more features than patient programmer 24. For example, clinician programmer 22 may be configured for more advanced programming features than patient programmer 24. This may allow a user to modify more therapy parameters with clinician programmer than with patient programmer 24. Patient programmer 24 may have a relatively limited ability to modify therapy parameter values with which IMD 16 generates electrical stimulation in order to minimize the possibility of patient 14 selecting therapy parameters that are harmful to patient 14. Similarly, clinician programmer 22 may conduct more advanced diagnostics of IMD 16 than patient programmer 24.

As described in further detail below, processor 70 of clinician programmer 22 may interrogate IMD 16 and/or patient programmer 24 to retrieve any collected information stored within memories 35 (FIG. 2), 42 (FIG. 3), such as information associated with therapy programs, which may include information received from patient 14 relating to a mood state, or information relating to sensed physiological parameter values. The sensed physiological parameters may include, for example, brain signals monitored at a respective one of two or more locations of a mood circuit of brain 12. Memory 72 of clinician programmer 22 may include software including instructions that cause processor 70 of clinician programmer 22 to periodically interrogate IMD 16 and/or patient programmer 24. Memory 72 may associate stored brain signals with therapy programs, such as therapy programs that defined the stimulation therapy delivered to patient 14 at the time the brain signals were sensed. The information relating to the therapy programs may be stored within therapy program information portion 80 of memory 72.

In general, during a programming session, a clinician may select values for a number of programmable therapy parameters in order to define the electrical stimulation therapy to be delivered by IMD 16 to patient 14. For example, the clinician may select a combination of electrodes 30, 31 carried by one or more implantable leads 20 (FIG. 2), and assign polarities to the selected electrodes. In addition, the clinician may select an amplitude, which may be a current or voltage amplitude, a pulse width, and a pulse rate, in the case of an IMD 16 that delivers stimulation pulses to patient 14. A group of parameter values, including electrode configuration (electrode combination and electrode polarity), amplitude, pulse width and pulse rate, may be referred to as a therapy program in the sense that they drive the neurostimulation therapy to be delivered to the patient.

Programs selected during a programming session using clinician programmer 22 may be transmitted to and stored within one or both of patient programmer 24 and IMD 16. Where the programs are stored in patient programmer 24, patient programmer 24 may transmit the programs selected by patient 14 to IMD 16 for delivery of neurostimulation therapy to patient 14 according to the selected program. Where the programs are stored in IMD 16, patient programmer 24 may receive a list of programs from IMD 16 to display to patient 14, and transmit an indication of the selected program to IMD 16 for delivery of neurostimulation therapy to patient 14 according to the selected program.

During a programming session, which may also be referred to as a therapy program trial session, the clinician may specify a program using clinician programmer 22 by selecting values for various therapy parameters. When a program is specified, the clinician may test the program by directing clinician programmer 22 to control IMD 16 to deliver therapy according to the program to patient 14. The clinician or patient may enter rating information into the programming device for each tested program. The rating information for a tested program may include information relating to effectiveness of delivery of stimulation therapy according to the program in treating symptoms of the patient, side effects experienced by the patient due to the delivery of stimulation therapy according to the program, or both. In the case of psychiatric disorder stimulation therapy, efficacy information may include an indication of patient mood state during therapy delivery and following therapy delivery. The patient mood state information may include, for example, patient feedback (received via patient programmer 22), brain signal information from two or more locations of a mood circuit, and/or one or more secondary indicators indicative of a particular patient mood state also monitored by system 10.

During the programming session, multiple therapy programs may be tested (or trialed). That is, during a programming session, IMD 16 may deliver therapy to patient 14 according to a first therapy program, followed by a second therapy program, and so forth, in order to assess the efficacy of each therapy program. Clinician programmer 22 may maintain a session log that includes a listing of programs tested on patient 14, rating information provided by the clinician or patient 14 for programs of the list, brain signal information from multiple locations along a mood circuit, and mood state information. The listing may be ordered according to the rating information in order to facilitate the selection of programs from the list by the clinician.

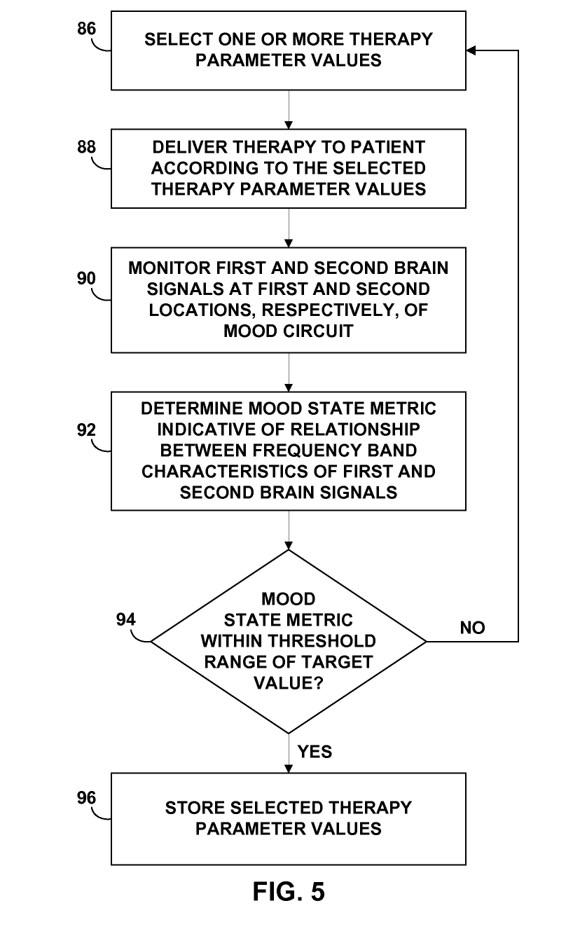

As previously described, in some examples, IMD 16 may monitor brain signals of patient 14 at two or more locations along a common mood circuit within brain 12, and control the delivery of therapy to brain 12 of patient 14 based on a relationship of the frequency band characteristics of the brain signals. FIG. 5 is a flow diagram illustrating an example technique for controlling therapy delivery to patient 14 based on activity within a mood circuit of brain 12 that is related to a patient mood state. The technique shown in FIG. 5 may be implemented to select one or more therapy parameter values that provide efficacious therapy to patient 14, e.g., to titrate therapy system 10. For example, the technique shown in FIG. 5 may be used to select one or more therapy programs that are stored in memory 35 (FIG. 2) of IMD 16, e.g., during a programming session. In addition, the technique shown in FIG. 5 may be implemented to control therapy delivery to patient 14 in a closed-loop manner.

While FIGS. 5-10, 12, 13A, and 13B are primarily described as being performed by processor 34 (FIG. 2) of IMD 16, in other examples, processor 40 (FIG. 3) of patient programmer 24, processor 70 (FIG. 4) of clinician programmer 22 or a processor of another device may perform any part of the techniques described herein.

A clinician, alone or with the aid of processor 34 (FIG. 2) of IMD 16, processor 40 (FIG. 3) of patient programmer 24, processor 70 (FIG. 4) of clinician programmer 22 or a processor of another device may select one or more initial therapy parameter values (86) that define the therapy delivery by IMD 16. The initial one or more therapy parameter values may be selected based on, for example, therapy parameters that are expected to provide patient 14 with efficacious therapy (e.g., stimulation therapy) to manage a psychiatric disorder. Thus, in some examples, the initial one or more therapy parameter values may be based on past therapy programming sessions for one or more patients that have a similar psychiatric disorder as patient 14.

IMD 16 may deliver therapy to patient 14 according to the selected therapy parameter values (88). IMD 16 may monitor a first brain signal at a first location of a mood circuit within brain 12 and a second brain signal at a second location of a mood circuit within brain 12 via sensing module 26 (FIG. 1) and/or sensing module 33 (FIG. 2) (90). In the example of FIG. 5, sensing module 26 and/or sensing module 33 of IMD 16 may monitor the first and second brain signals at first and second locations along the same mood circuit of brain 12. However, the same or similar technique may be incorporated in any example in which IMD 16 monitors brain signals at more than two locations along the same brain circuit of patient 14.

As previously described, a mood circuit may generally refer to regions of a brain functionality related to one another via neurological pathways in a manner that causes activity within the respective regions of a common brain circuit to be influenced at least in part based on the mood state of a patient. In some examples, depending on the regions of the brain included in a mood circuit, the regions of brain 12 included in a mood circuit may allow the first and second brain signals to be monitored at different locations within the same hemisphere of brain 12. In other examples, the regions of brain 12 included in a mood circuit may allow the first and second brain signals to be monitored in different hemispheres of brain 12.

In some example, the first and second brain signals may be monitored at different locations within the brain structure, while in other examples, the first and second brain signals may be monitored at locations that are in brain structures that are a part of the same mood circuit. Examples of brain structures include, for example, the internal capsule, the cingulate cortex, the prefrontal cortex, the orbitofrontal cortex, the medial or nucleus accumbens, thalamus, intralaminar thalamic nuclei, amygdala, hippocampus, the lateral hypothalamus, the Locus ceruleus, the dorsal raphe nucleus, ventral tegmentum, the substantia nigra, subthalamic nucleus, the inferior thalamic peduncle, the dorsal medial nucleus of the thalamus, and areas thereof.

The brain structures that are included in a mood circuit may be unique to different psychiatric disorders. For example, the regions of brain 12 included in a depressive mood circuit may include one or more regions of brain 12 that are not included in an obsessive compulsive disorder mood circuit. Furthermore, in some examples, the regions of brain 12 included in a mood circuit may be patient specific. For example, one or more regions of brain 12 included in a depressive mood circuit of a first patient may be different than that of a depressive mood circuit in a second patient.

In some examples, brain regions of a depressive mood circuit may include the medial frontal cortex, the full extent of the anterior and posterior cingulate, medial temporal lobe, dorsal medial thalamus, hypothalamus, nucleus accumbens, the dorsal brainstem, and combinations thereof. As another example, brain regions of another depressive mood circuit may include frontal pole, medial temporal lobe, cerebellum, nucleus accumbens, thalamus, hypothalamus, the brainstem, and combinations thereof. Accordingly, sensing module 26 and/or sensing module 33 may monitor the first and second signals at first and second locations of one or more of the regions included in a respective mood circuit.

Processor 34 (FIG. 2) of IMD 16 may determine a value indicative of a relationship between a first frequency band characteristic of the first brain signal and a second frequency band characteristic of the second brain signal (92). This value may be referred to as a “mood state metric.” As previously indicated, in some examples, the frequency band characteristics may comprise the power level within a selected frequency band of the first and second brain signals. For example, it may be determined that the amount of power in a certain frequency band monitored at the first location relative to the amount of power in a same frequency band monitored at the second location may be indicative of a mood state of patient 14. In such a case, sensing module 26 and/or sensing module 33 may be configured to monitor the power of both the signals within the same frequency band. In other examples, the frequency band characteristics may comprise the power level of the first and second brain signals within different frequency bands. For example, it may be determined that the amount of power in a certain frequency band monitored at the first location relative to the amount of power in a different frequency band monitored at the second location may be indicative of a mood state of patient 14. In such a case, sensing module 26 and/or sensing module 33 may be configured to monitor the power of both signals at the different respective frequency bands.

The relative power within one or more selected frequency bands of the first and second brain signals may be an indicator of the mood state of patient 14. Thus, in some examples, the mood state metric, which is indicative of the relationship between the first and second frequency characteristics, may comprise a ratio of a first power level of the first brain signal in a selected frequency band (e.g., an alpha band) to a second power level of the second brain signal in the selected frequency band. This ratio may be indicative of a current mood state that patient 14, e.g., the mood state that coincides in time with the sensing of the first and second brain signals. In other examples, the mood state metric may comprise a difference between the first and second power levels. In some examples, the mood state metric comprise a difference between the first and second power levels over the sum of the first and second power levels. The first and second power levels may be determined from the same or substantially similar frequency bands of the brain signal at each location and/or from different frequency bands of the brain signal at each location.

The frequency band of the signals monitored by sensing module 26 (FIG. 1) and/or sensing module 33 of IMD 16 (FIG. 2) at each respective brain location of the mood circuit may depend the psychiatric disorder of patient 14. Different frequency bands may be associated with different activity in brain 12. One example of the frequency bands is shown in Table 1:

TABLE 1

| Frequency (f) Band | |
|---|---|
| Hertz (Hz) | Frequency Information |
| f < 5 Hz | δ (delta frequency band) |
| 5 Hz ≦ f ≦ 13 Hz | α (alpha frequency band) |
| 13 Hz ≦ f ≦ 30 Hz | β (beta frequency band) |
| 50 Hz ≦ f ≦ 100 Hz | γ (gamma frequency band) |
| 100 Hz ≦ f ≦ 200 Hz | high γ (high gamma frequency band) |

It is believed that some frequency band components of a brain signal (e.g., an EEG signal or ECoG signal) may be more revealing of particular mood states than other frequency components. For example, the EEG signal activity within the alpha band may attenuate with mood states associated with a MDD disorder of patient 14. Thus, the range of the frequency band of the brain signals monitored by sensing module 26 and/or sensing module 33 at each respective brain location may depend on the psychiatric disorder of patient 14. For example, it may be determined that the amount of power in an alpha frequency band ranging from about 8 Hz to about 10 Hz of a first brain signal monitored at the first location relative to the amount of power in the alpha frequency band of a second brain signal monitored at the second location may be indicative of a positive mood state of patient 14. In such a case, sensing module 26 may be configured to monitor the power of the respective signal in the frequency bands corresponding to a positive mood state of patient 14.

Processor 34 may determine whether the mood state metric is within a threshold range of a target value (94). The target value may comprise, for example, a value indicative of a relationship between the first and second frequency characteristics when patient 14 is in a positive mood state. For example, the target value may be indicative of a mood state in which the psychiatric disorder of patient 14 is considered to be managed, and, thus, therapy delivery to patient 14 is considered efficacious. The target value may be stored in memory 35 of IMD 16 or a memory of another device (e.g., one or both programmers 22, 24) and communicated to IMD 16.

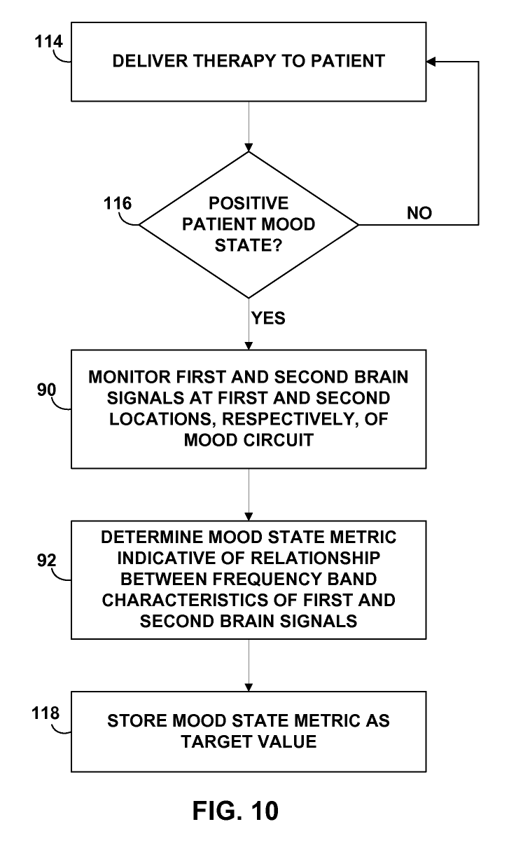

The target value may be determined using any suitable technique. In some examples, the target value may be specific to a positive mood state of patient 14 or may be general to more than one patient. For example, in some examples, the target value may be defined based on observations of two or more patients, e.g., one or more patients exhibiting the same or similar mood disorder to that of patient 14 and/or receiving similar therapy to that of patient 14. A positive mood state may be relative. For example, if patient 14 has a MDD and therapy system 10 provides therapy to improve the patient's depressive mood, a positive mood state would be a relatively less depressed mood state than the patient's baseline mood state. Alternatively, the positive mood state may be an objectively positive mood state, rather than a relatively positive mood state. For example, although a moderately depressed mood state may be an improvement on the patient's baseline mood state, a moderately depressed mood state may not be a positive mood state, but rather, a substantially non-depressed mood state may be a positive mood state. An example technique for determining the target value is described with respect to FIG. 10.

The threshold range may be stored in memory 35 of IMD 16 or a memory of another device (e.g., one or both programmers 22, 24). A clinician may select the threshold range. In some examples the threshold range may comprise, for example, about 75% to about 100%. Thus, if the mood state metric indicative of the relationship between the first and second frequency characteristics determined by processor 34 is about 75% to about 100% of the target value, processor 34 may determine that the mood state metric is within the threshold range of the target value. Other threshold ranges are contemplated and may be specific to patient 14 and the psychiatric disorder with which patient 14 is afflicted.

If the mood state metric is within the threshold range of the target value (94), processor 34 may determine that therapy delivery to patient 14 according to the one or more therapy selected therapy parameter values provided efficacious therapy to patient 14. Efficacious therapy may indicate, for example, that the patient's mood state was improved or maintained at an acceptable mood state. Processor 34 may then store the one or more therapy selected therapy parameter values in memory 35, e.g., as a therapy program for therapy delivery to patient 14 on a chronic (e.g., non-permanent) basis.

On the other hand, if the mood state metric is not within the threshold range of the target value (94), e.g., because the mood state metric differs from the target value by a threshold amount (which may be stored in memory 35 of IMD 16 as a threshold value), processor 34 may select one or more additional therapy parameter values (86) and test the therapy delivery according to the one or more additional therapy parameter values using the technique shown in FIG. 5. The technique shown in FIG. 5 may be implemented until one or more efficacious therapy programs are identified for patient 14.

In some examples, the mood state metric indicative of the relationship between the frequency band characteristics of the first and second brain signals sensed within a mood circuit of brain 12 may be used to control therapy delivery in a substantially closed-loop manner.

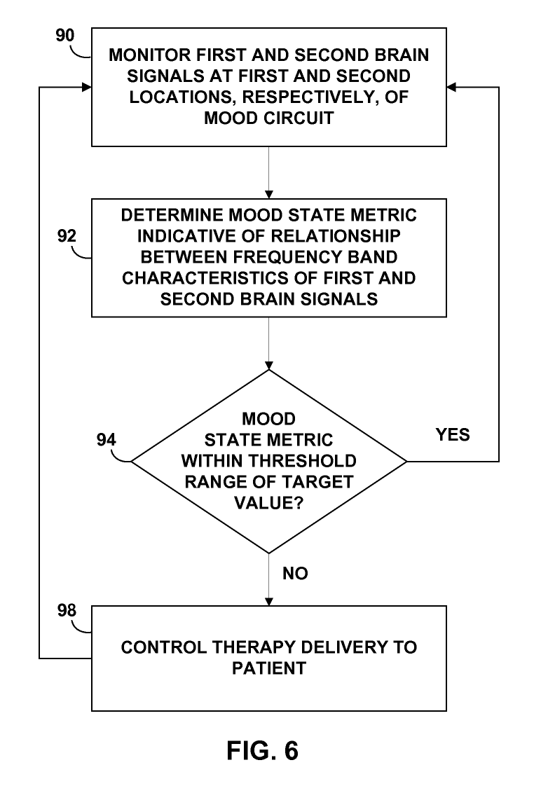

FIG. 6 is a flow diagram illustrating an example technique for controlling therapy delivery to patient 14 based on first and second brain signals sensed within a mood circuit of brain 12. IMD 16 may monitor a first brain signal at a first location of a mood circuit within brain 12 and a second brain signal at a second location of a mood circuit within brain 12 via sensing module 26 (FIG. 1) and/or sensing module 33 (FIG. 2), as described above with respect to FIG. 5 (90). Processor 34 (FIG. 2) of IMD 16 may determine a mood state metric indicative of a relationship between a first frequency band characteristic of the first brain signal and a second frequency band characteristic of the second brain signal (92). Processor 34 may determine whether the mood state metric is within a threshold range of a target value (94).

If the mood state metric is within a threshold range of a target value (94), processor 34 may not modify therapy delivery to patient 14 and may continue monitoring the first and second brain signals (90). For example, processor 34 may determine that patient 14 is in an acceptable mood state if the mood state metric is within a threshold range of the target value. Accordingly, modifications to therapy delivery to patient 14 may not be necessary to manage the patient's mood state.

On the other hand, if the mood state metric is not within a threshold range of a target value (94), processor 34 may determine that patient 14 is not in an acceptable mood state and modification to the therapy delivery to patient 14 is desirable. Accordingly, processor 34 may control therapy delivery to patient (98) if the mood state metric is not within a threshold range of the target value. IMD 16 may deliver stimulation therapy to brain 12 of patient 14 according to a therapy program via leads 20 to manage a mood disorder of patient 14. Managing a mood disorder may include, for example, decreasing or even eliminating the severity of symptoms associated with the patient mood state (e.g., a depressive mood state or a manic mood state).

In some examples, IMD 16 may provide therapy to regions of brain 12 within the same mood circuit as the first and second locations in which sensing module 26 and/or sensing module 33 monitors the first and second brain signals. For example, this may be the case in configurations in which sense electrodes of sensing module 26 and/or sensing module 33 are located on the same leads as stimulation electrodes 30, 31 (FIG. 2) used to deliver therapy to patient 14. In some examples, at least some of stimulation electrodes 30, 31 may also function as sense electrodes. In other examples, IMD 16 may deliver therapy to regions of brain 12 outside of the mood circuit or in a different part of the mood circuit than the region in which sensing module 26 and/or sensing module 33 senses brain signals.

Processor 34 may control the delivery of therapy to patient 14 (98) by adjusting one or more of the stimulation parameter values if the patient's mood state has changed from a prior detected mood state, as indicated by the mood state metric that is not within a threshold range of the target value. In this manner, processor 34 may titrate one or more parameter values of the therapy based on a detected change in the patient's mood state. In some examples, the frequency with which processor 24 may adjust to the therapy parameter values with which signal generator 37 of therapy module 32 (FIG. 2) of IMD 16 generates stimulation parameter values may be limited to a predetermined frequency. This may help limit the changing of stimulation parameter values at a rate that exceeds the rate at which brain 12 may react to the therapy.

Many therapy systems that provide stimulation therapy to patient 14 to manage a psychiatric disorder provide substantially continuous delivery of stimulation to patient 14. One drawback with the continuous stimulation approach is the inefficient use of power. For example, with continuous delivery, therapy may be provided to patient 14 even though patient 14 does not need the therapy. Therapy may be unnecessary or undesired when patient 14 is in a positive mood state. Accordingly, in some examples, therapy system 10 may be configured to deliver therapy to patient 14 only at a time when patient 14 needs therapy, e.g., when patient 14 is not in a positive mood state, rather than in a substantially continuous manner. For example, processor 34 may control the delivery of therapy to patient 14 (98) by initiating the delivery of the therapy to patient 14.

In some cases, processor 34 may initiate the delivery of therapy to patient 14 when the mood state metric indicative of the relationship between the first and second frequency characteristics indicates a negative mood state (e.g., a depressed state, hypomanic state or manic state). In other examples, processor 34 may not determine a specific mood state associated with the mood state metric, but may instead initiate therapy delivery to patient 14 in order to maintain the patient's brain activity at a particular level, e.g., as indicated by a mood state metric that is within the threshold range of the target value. As described above, the target value may be associated with a positive patient mood state, or at least be indicative of a mood state in which therapy delivery to patient 14 is unnecessary. Thus, if the mood state metric is not within the threshold range of the target value, processor 34 may initiate therapy delivery in order to attempt to manage the patient's psychiatric disorder.

In some cases, IMD 16 may deliver the therapy to patient 14 for a period of time that is appropriate to successfully manage the mood disorder of patient 14, e.g., by driving the mood state of patient 14 to a positive mood state. After that period of time, processor 34 may terminate the delivery of therapy to patient 14 although sensing module 26 and/or sensing module 33 may continue to monitor the first and second brain signals (90) to detect a change in the patient's mood state that may require therapy delivery or an adjustment to therapy delivery. In this manner, IMD 16 may be configured to only deliver therapy to patient 14 when appropriate, rather than in a continuous manner without respect to patient mood state.

In some examples, therapy may be delivered prior to monitoring the first and second brain signals (90). Accordingly, controlling the delivery of therapy to patient 14 may include terminating delivery of the therapy to patient 14. In some examples, when the mood state metric is within the threshold range of the target value, processor 34 may determine that therapy delivery to patient 14 was efficacious such that, for example, patient 14 is in a positive mood state. Because the effects of the therapy delivery to patient 14 may persist after IMD 16 stops actively delivering stimulation to brain 12, processor 34 may suspend (or terminate) the delivery of therapy to patient 14 and the patient's positive mood state may be maintained. Processor may continue monitoring the first and second brains signals at different portions of the mood circuit (90) after therapy is terminated.

In some examples, processor 34 may control the delivery of therapy to patient 14 (98) by at least maintaining therapy delivery according to a currently selected therapy program or switching therapy programs that define the stimulation therapy delivered to patient 14. In some examples, if IMD 16 was delivering therapy to patient 14 according to a first therapy program prior to the determination of whether the mood state metric indicative of the relationship between the first and second frequency characteristics is within the threshold range of the target value, IMD 16 may continue the delivery of therapy to patient 14 according to the first therapy program if the mood state metric is within the threshold range of the target value (94). For example, processor 34 may determine that the first therapy program provides efficacious therapy to patient 14 and that therapy delivery to patient 14 may be maintained.

In other examples, if IMD 16 was delivering therapy to patient 14 according to a first therapy program prior to the determination of whether the mood state metric is within the threshold range of the target value, IMD 16 may control therapy to patient (98) by delivering therapy to patient 14 according to a second therapy program, where the second therapy program comprises at least one different stimulation parameter value than the first therapy program. For example, processor 34 may determine that patient 14 is in a positive mood state (e.g., an improved mood state), and, accordingly, the intensity of therapy may be decreased. An intensity of stimulation may be related to the current or voltage amplitude of a stimulation signal, a frequency of the stimulation signal, and, if the signal comprises a pulse, a pulse width, burst pattern or pulse shape of the stimulation signal.

It has also been found that patient 14 may adapt to DBS provided by IMD 16 over time. That is, a certain level of electrical stimulation provided to brain 12 may be less effective over time. This phenomenon may be referred to as “adaptation.” As a result, any beneficial effects to patient 14 from the DBS may decrease over time. While the electrical stimulation levels (e.g., amplitude of the electrical stimulation signal) may be increased to overcome such adaptation, the increase in stimulation levels may consume more power, and may eventually reach undesirable or harmful levels of stimulation.

When therapy parameter values are modified upon the detection of a positive mood state, e.g., based on a comparison of the value indicative of the relationship between the first and second frequency characteristics to the target value (94), the rate at which patient adaptation to the therapy, whether electrical stimulation, drug delivery or otherwise, may decrease. Thus, therapy system 10 enables the therapy provided to patient 14 via IMD 16 to be more effective for a longer period of time as compared to systems in which therapy is delivered continuously or substantially continuously to patient 14 regardless of the patient mood state.

In some examples, processor 34 may also consider the change in the frequency band characteristics of the first and second signals over time in order to control therapy delivery to patient 14. For example, if processor 34 associates a ratio of power levels within one or more frequency bands of the first and second brain signals with a mood state, processor 34 may determine whether the frequency band characteristics of the first and second signals are converging toward each other, diverging away from each other, or approximately constant over time. Processor 34 may consider such information when determining the adjustments to therapy delivery to patient 14 that are made based on the first and second brain signals. For example, processor 34 may maintain therapy delivery according to a particular therapy program if the change in the frequency band characteristics of the first and second signals over time indicates that a detected positive mood state of patient 14 is relatively stable, e.g., when the power ratio of the frequency band characteristics of the first and second brain signals is relatively consistent over time.

In some examples, processor 34 may include a buffer to track the change in the first and second frequency band characteristics over time. A separate buffer may be used for the first frequency band characteristic and the second frequency band characteristic. The buffer may be useful for indicating whether the first frequency band characteristic is increasing over time or decreasing over time, and whether the second frequency band characteristic is increasing over time or decreasing over time. The buffer may include, for example, a circular buffer. If the first and second frequency band characteristics each comprise a power level of the respective brain signal in a specific frequency band, a time-averaged power level may be inserted into the buffer. This may generate a rolling history of the power levels of the respective brain signal over time, which processor 34 may use to evaluate slope for the power level of the respective brain signal in the selected frequency band over time.

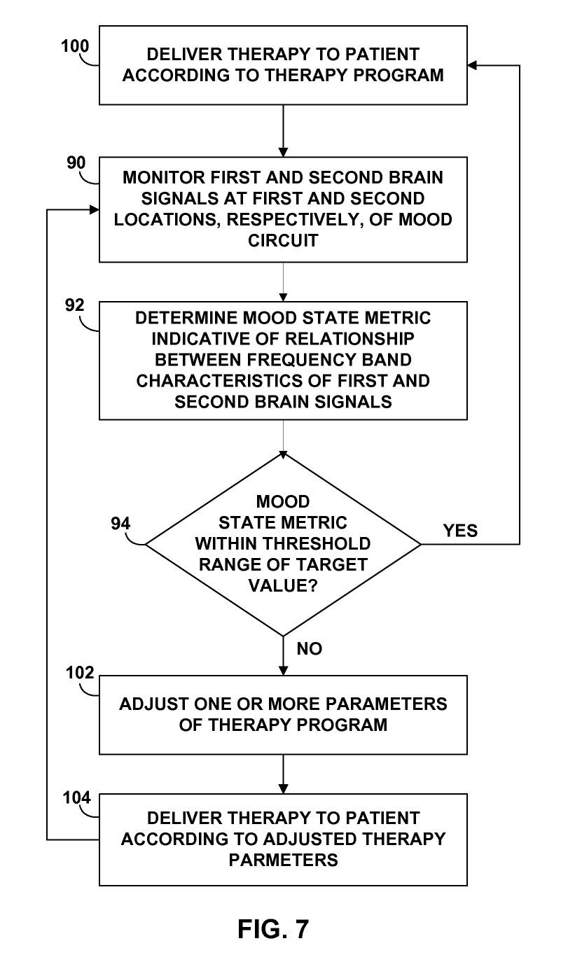

FIG. 7 is a flow diagram of an example technique for controlling therapy delivery to patient 14, e.g., in a closed-loop manner, based on brain signals sensed within different parts of a mood circuit. IMD 16 may deliver therapy to patient 14 according to a therapy program (100). IMD 16 may monitor a first brain signal at a first location of a mood circuit within brain 12 and a second brain signal at a second location of a mood circuit within brain 12 via sensing module 26 (FIG. 1) and/or sensing module 33 of IMD 16 (FIG. 2), as described above with respect to FIG. 5 (90). Processor 34 (FIG. 2) of IMD 16 may determine a mood state metric indicative of a relationship between a first frequency band characteristic of the first brain signal and a second frequency band characteristic of the second brain signal (92). Processor 34 may determine whether the mood state metric is within a threshold range of a target value (94).

If the mood state metric is within the threshold range of the target value, processor 34 may determine that the therapy parameter values defined by the therapy program provide efficacious therapy to patient 14. That is, if the mood state metric is within the threshold range of the target value, processor 34 may determine that that the therapy being delivered to patient 14 is successfully managing the mood disorder of patient 14, and that the parameters defined by therapy program are appropriate. Accordingly, processor 34 may control signal generator 37 (FIG. 2) of IMD 16 to continue delivering therapy to patient according to the therapy program (100).

If the mood state metric is not within the threshold range of the target value (94), processor 34 may determine that the therapy parameter values defined by the therapy program may not be providing patient 14 with efficacious therapy, and, accordingly, processor 34 may adjust one or more therapy parameter values (102). For example, in the case of a therapy program that defines stimulation parameter values, processor 34 may modify an amplitude or frequency of the stimulation signal, or, in the case of stimulation pulses, the pulse width, pulse rate, and burst pattern of the stimulation signal.

Processor 34 may modify the one or more therapy parameter values using any suitable technique. In some examples, processor 34 may modify a specific stimulation parameter value defined by the therapy program according to a set of rules stored in memory 35. The rules may define an acceptable range of values for the specific stimulation parameter that provide efficacious therapy to patient 14 and/or are not harmful to patient 14. In addition, the rules may define the increments and frequency with which processor 34 may modify the stimulation parameter value. Other types of rules for controlling the modification to one or more stimulation parameter values are also contemplated.

Processor 34 may be configured to communicate information to a clinician and/or patient relating to the relative influence that the certain parameter adjustments have on a patient mood state. For example, although certain adjustment to one or more parameters may not result in successfully changing the mood state indicated by mood state metric, in some cases, adjustments to the parameters may cause the first and second signals to converge towards or diverge from the target value. For example, in the case of a target value that indicates a ratio of the power level of the first brain signal in a particular frequency band to the power level of the second brain signal that is indicative of a positive mood state, one or more therapy parameter adjustments may cause the mood state metric to converge toward the target value, but not come within the threshold range of the target value. Alternatively, one or more parameter adjustments may cause the mood state metric to diverge from the target value. Processor 34 may be configured to determine and communicate this information to the clinician via programmer 22 to provide guidance in adjusting therapy parameters.

In other examples, processor 34 may modify the one or more therapy parameter values by switching therapy programs that define the therapy. For example, if a plurality of therapy programs are stored in memory 35 of IMD 16 or a memory of another device (e.g., programmers 22 or 24), processor 34 may discontinue therapy according to a first therapy program and deliver therapy to patient 14 according to a second stored therapy program. The therapy programs may be stored in a specific order, e.g., a specific order of based on intensity of therapy, power consumption, or a likelihood that the therapy delivery according to the respective therapy program may provide efficacious therapy to patient.

After modifying the one or more therapy parameter values (102), processor 34 may deliver therapy to patient 14 according to the adjusted therapy parameter values (104). In order to determine whether the adjustment to the therapy parameter values was effective in increasing managing the patient's mood disorder, processor 34 may continue monitoring first and second brain signals within different parts of a mood circuit (90), e.g., via sensing module 26 and/or sensing module 33. Processor 34 may, for example, determine that the adjustment to the therapy parameter values was effective if the mood state metric indicative of the relationship between the first and second frequency band characteristics of the first and second brain signals, respectively, is within a threshold range of the target value (94).

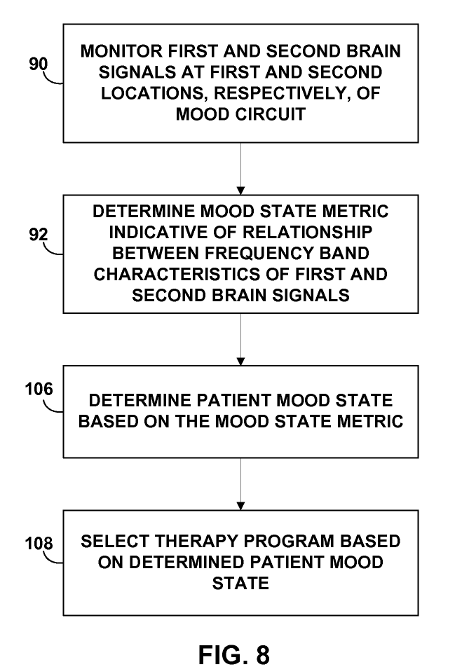

FIG. 8 is a flow diagram illustrating an example technique processor 34 of IMD 16 or a processor of another device may implement in order to control therapy delivery to patient 14 based on a detected mood state. The technique shown in FIG. 8 may be used, for example, to control the stimulation parameter values with which signal generator 37 (FIG. 2) generates and delivers therapy to patient 14. Processor 34 of IMD 16 may monitor a first brain signal at a first location of a mood circuit within brain 12 and a second brain signal at a second location of a mood circuit within brain 12 via sensing module 26 and/or sensing module 33 (FIG. 1), as described above with respect to FIG. 5 (90). Processor 34 (FIG. 2) of IMD 16 may determine a mood state metric indicative of a relationship between a first frequency band characteristic of the first brain signal and a second frequency band characteristic of the second brain signal (92).

Processor 34 may determine a patient mood state based on the mood state metric (106). In some examples, memory 35 of IMD 16 or a memory of another device may store a plurality of mood state metrics and associated patient mood states. Processor 34 may then reference the stored information to determine, based on the mood state metric, the patient mood state. The brain activity within different parts of a mood circuit, as indicated by frequency band characteristics of brain signals monitored within portions of the brain associated with the mood circuit, may be indicative of a patient mood state. For example, if patient 14 is in a depressive mood state, a power level within a particular frequency band of a first brain signal sensed at a first part of the mood circuit may be greater than a power level within the same frequency band or a different frequency band of a second brain signal sensed at a second (and different) part of the mood circuit. This difference in power levels may be characterized as a ratio of the power levels or a difference in the power levels may be indicative of the patient mood state. During a trial stage, a clinician may determine a patient mood state and the ratio or other values indicative of the relationship between the power levels within the particular frequency bands of the first and second brain signals sensed at the time the mood state was determined. The ratio or other values may then be associated with the patient mood state in memory 35 of IMD 16.

After determining the patient mood state (106), processor 34 may select a therapy program based on the determined mood state (108). Processor 34 may control signal generator 37 (FIG. 2) to generate and deliver therapy to patient 14 according to the selected therapy program. As indicated above, in some examples, memory 35 (FIG. 2) of IMD 16 or a memory of another device may store a plurality of therapy programs that are associated with one or more mood states. The therapy parameter values of the therapy program may be selected to provide efficacious therapy to patient 14 to manage the mood state associated with the therapy program. For example, memory 35 may store a first therapy program associated with a depressive mood state and a second therapy program associated with a manic mood state. The first therapy program may be configured to transition patient 14 from the depressive mood state to a mood state with less severe depression symptoms. The second therapy program may be configured to transition patient 14 from the manic mood state to a non-manic mood state.

In some examples, depending on the patient psychiatric disorder, IMD 16 may deliver therapy to patient 14 to manage a single mood state, such as an obsessive-compulsive mood state in which patient 14 is afflicted with obsessive thoughts or related compulsions.

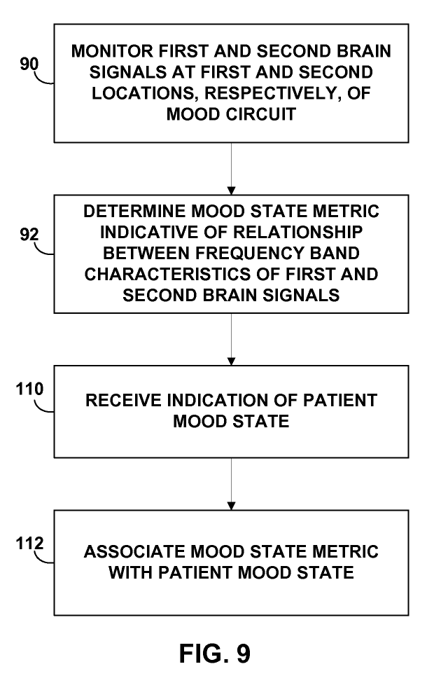

FIG. 9 is a flow diagram illustrating an example technique for associating a mood state metric with a particular patient mood state. The patient mood state may be, for example, a positive mood state that indicates patient 14 is in a condition in which therapy delivery to patient 14 is efficacious or not necessary. In other examples, the patient mood state may be a negative mood state, such as a depressive mood state, manic mood state, or obsessive-compulsive mood state. Thus, in some examples, the patient mood state may be used to select one or more therapy parameters for therapy delivery to patient 14.

Processor 34 of IMD 16 may monitor a first brain signal at a first location of a mood circuit within brain 12 and a second brain signal at a second location of a mood circuit within brain 12 based on brain signals sensed by sensing module 26 (FIG. 1) and/or sensing module 33 (FIG. 2), as described above with respect to FIG. 5 (90). Processor 34 (FIG. 2) of IMD 16 may determine a mood state metric indicative of a relationship between a first frequency band characteristic of the first brain signal and a second frequency band characteristic of the second brain signal (92), as described above with respect to FIG. 5. Processor 34 may also receive an indication of patient mood state (110) at a time that generally coincides with the sensing of the first and second brain signals (90). This may help processor 34 determine the mood state of patient 14 at the time in which the brain signals are sensed. The time period that generally coincides with the sensing of the first and second brain signals may include, for example, a time period of about one second to about one minute or more prior to the monitoring of the brain signals and one second to about one minute or more after the monitoring of the brain signals.

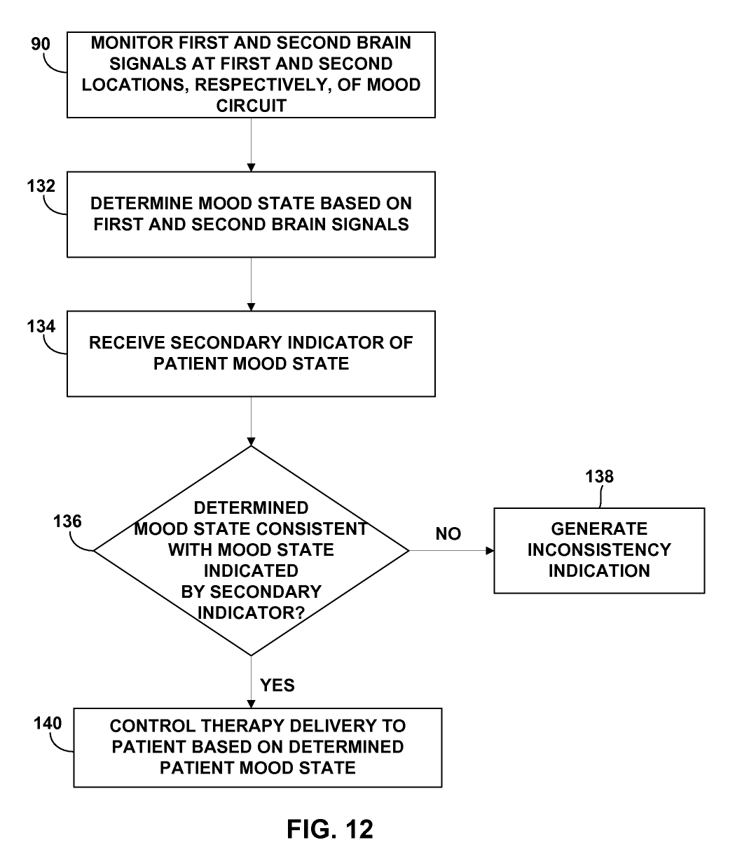

Other indicators of patient mood state, e.g., based on physiological parameters of patient 14 instead of in addition to brain signals, may be used along with the user input or in place of the user input. These indicators may be referred to as “secondary” indicators are described below with respect to FIG. 12.

In some examples, processor 34 may receive an indication of patient mood state (110) from input from a user, e.g., from a clinician or patient 14. The clinician may provide mood state input via clinician programmer 22 (FIG. 1) and patient 14 or a caretaker of patient 14 may provide mood state input via patient programmer 24 (FIG. 1). In some examples, the clinician may gather a relatively objective evaluation of the patient's mood state based on surveying the patient's mood state. As examples, a clinician's indication of patient mood state may be based on a patient's response to various questions, such as, e.g., the Beck Depression Inventory, Hamilton Rating Scale for Depression (HAM-D) or the Montgomery-Asberg Depression Rating Scale (MADRS), in examples in which the mood disorder of patient 14 is MDD. The Beck Depression Inventory and the HAM-D are both 21-question multiple choice surveys that is filled out by patient 14, and the MADRS is a ten-item questionnaire. The answers to the questions may indicate the severity of patient symptoms or the general patient mood state.

As another example, the clinician may evaluate the patient's mood state using the Yale-Brown Obsessive Compulsive Scale (Y-BOCS), which may be appropriate in cases in which the patient's mood disorder is OCD, as the Y-BOCS may be used as a test to rate the severity of OCD symptoms. The Y-BOC scale is a clinician rated, ten item scale in which each item is rated from 0 (no symptoms) to 40 (extreme symptoms) based at least in part on patient answers to questions related to the patient's mood disorder.

Additionally or alternatively, in some examples, patient mood state may be indicated by a patient 14, e.g., via clinician programmer 22 or patient programmer 24, based on the patient's subjective assessment of their mood state. For example, patient 14 may provide a subject assessment of mood state based on severity of symptoms, which may be rated on a scale (e.g., a 1 to 10 scale, whereby 1 indicates relatively mild symptoms and 10 indicates relatively severe symptoms). The self-rating of mood state by patient 14 may be more subjective than the mood state indication provided by a clinician.

Processor 34 may associate the mood state metric with the patient mood state and store the information in memory 35 (FIG. 2) of IMD 16 or a memory of another device (112). In addition, in some examples, processor 34 may store the first and second brain signals that were monitored at the time the mood state was determined, As described with respect to FIG. 8, the mood state metric and the associated patient mood state information may be useful for controlling therapy delivery to patient 14, e.g., in a closed-loop manner. For example, processor 34 may select a therapy program that defines the stimulation generated by signal generator 37 (FIG. 2) of IMD 16 based on a detected mood state. As described above, the mood state may be detected based on brain signals sensed within different parts of a mood circuit, e.g., based on a mood state metric that is indicative of a relationship between a first frequency band characteristic of the first brain signal and a second frequency band characteristic of the second brain signal.

Processor 34 may periodically (e.g., on a daily, weekly or monthly basis) perform the technique shown in FIG. 9 in order to reliably maintain a relationship between a mood state metric and a patient mood state. Patient programmer 24 or clinician programmer 22 may periodically prompt patient 14 to provide input indicating a patient mood state and correlate sensed brain signals and/or a mood state metric with the patient mood state. Brain 12, and, in particular, a mood circuit within brain 12, may change over time, such that the mood state metric that is indicative of a particular mood state of patient 14 may change over time. Thus, periodically reevaluating the mood state metrics that indicate a patient mood state may be useful.

FIG. 10 is a flow diagram illustrating an example technique for determining a target value for a mood state metric, which may be used to control therapy delivery to patient 14, e.g., as described with respect to FIGS. 5 and 6. The technique shown in FIG. 10 may be implemented during a trial stage in which a clinician adapts therapy system 10 to patient 14. The target value may be selected to be specific to patient 14 or may be selected for a class of patients that are afflicted with similar psychiatric disorders. Thus, the technique shown in FIG. 10 may be implemented for more than one patient in order to determine a target value. For example, the target values determined for a plurality of patients may be averaged or a median target value determined for the plurality of patients may also be selected as the target value used to track a patient mood state.

In accordance with the technique shown in FIG. 10, processor 34 of IMD 14 may control signal generator 37 (FIG. 2) of IMD 16 to deliver therapy to patient 14 (114). For example, signal generator 37 may generate and deliver therapy according to a therapy program that has been successful in modifying the mood state of patient 14 and/or other patients exhibiting the same mood disorder.

Processor 34 may determine whether patient 14 is in a positive mood state (116), e.g., based on factors other than brain signals. For example, processor 34 may receive an indication that patient 14 is in a positive mood state from a user, such as the clinician or patient 14. The user may interact with user input mechanism 56 (FIG. 3) of patient programmer 24 or user input mechanism 56 (FIG. 4) of clinician programmer 22 to provide input indicating patient 14 is in a positive mood state, and processor 40, 70 of programmer 24, 22, respectively, may transmit the positive mood state indication to IMD 16. The positive mood state indication may be, for example, transmitted to IMD 16 via the respective telemetry modules 38, 46, 76. In other examples, processor 34 may receive an indication that patient 14 is in a positive mood state based on one or more physiological parameters that are monitored by sensing module 26 and/or sensing module 33. The physiological parameters may include brain signals, but may not necessarily include brain signals.

If processor 34 determines that patient 14 is not in a positive mood state (116), e.g., because processor 34 has not received patient input or input from sensing module 34 from which processor 34 may determine patient 14 is in a positive mood state, processor 34 may continue controlling signal generator 37 (FIG. 2) of IMD 16 to deliver therapy to patient 14 (114). If the patient's mood state is not positive, signal generator 37 may continue to deliver therapy to the patient according to the same therapy program. For example, there may be a lag time between the delivery of the therapy to patient and a change in the patient's mood state to a positive mood state. In other examples, processor 34 may modify one or more therapy parameter values with which signal generator 37 generates the therapy delivered to patient 14 in order to achieve the positive patient mood state.

If processor 34 determines that patient 14 is in a positive mood state (116), processor 90 may monitor a first brain signal at a first location of a mood circuit within brain 12 and a second brain signal at a second location of a mood circuit within brain 12 via sensing module 26 and/or sensing module 33, as described above with respect to FIG. 5 (90). Processor 34 (FIG. 2) of IMD 16 may determine a mood state metric indicative of a relationship between a first frequency band characteristic of the first brain signal and a second frequency band characteristic of the second brain signal (92). In some examples, the clinician may select the frequency bands for determining the first and second frequency band characteristics and program processor 34, while in other examples, processor 34 may automatically select the frequency bands.

In some examples, sensing module 26 and/or sensing module 33 may sense initial brain signals at first and second locations of brain 12 that are a part of a common mood circuit prior to therapy delivery (114), e.g., when patient 14 is known to be in a negative mood state. Sensing module 26 and/or sensing module 33 may monitor the initial brain signals at relatively large frequency band, e.g., from about 5 Hz to about 250 Hz, to capture a wide band sample of the first and second brain signals. After determining patient 14 is in a positive mood state (116), sensing module 26 and/or sensing module 33 may monitor a relatively large frequency band, e.g., from about 5 Hz to about 250 Hz, of the first and second brain signals within the same parts of the mood circuit in which the initial brain signals were monitored (90) in order to capture a wide band sample of the first and second brain signals.

Processor 34 may analyze a spectrogram or a pseudo-spectrogram of the initial brain signals, as well as the first and second brain signals to determine whether the brain activity within a particular frequency band exhibited a discernable change after therapy delivery to improve the patient's mood state. A spectrogram provides a three-dimensional plot of the energy of the frequency content of a brain signal as it changes over time. A pseudo-spectrogram may be indicative of the energy of the frequency content of the brain signal within a particular window of time. The frequency band in which the first and second brain signals exhibited a discernable change compared to the initial brain signals (sensed prior to therapy delivery) may be selected as the frequency band of interest. The first and second frequency band characteristics may include the power level of the first and second brain signals within the frequency band of interest.

Processor 34 may store the mood state metric as a target value (118), e.g., for use in the technique described with respect to FIGS. 5 and 6. The target value may be a measure with which IMD 16 controls therapy delivery to patient 14. For example, as described above, IMD 16 may deliver therapy to patient 14 until a determined mood state metric is within a threshold range of the target value.

In addition, in some examples, processor 34 may store the first and second frequency band characteristics of the sensed brain signals within memory 35, and, in some cases, the first and second brain signals may also be stored in memory 35. In some cases, the mood state metric may be indicative of patient mood state. In addition, in some cases, the change in the first and second frequency band characteristics over time may be also be used to control therapy delivery to patient 14. For example, the change in the first and second frequency band characteristics over time may be used to detect a mood state in which therapy delivery to patient 14 may be beneficial. As another example, the change in the first and second frequency band characteristics over time may be used to suspend therapy delivery, e.g., because a positive mood state is detected for some period of time.

As noted above, although the techniques shown in FIGS. 9 and 10 are described as being performed by processor 34 of IMD 16, in other examples, a processor of another device (e.g., processor 40 of patient programmer 24 or processor 70 of clinician programmer 22) may perform any part of the techniques shown in FIGS. 9 and 10. For example, a clinician may utilize clinician programmer 22 to perform the association techniques of FIG. 9 during a trial stage in which the patient's condition is evaluated and one or more therapy programs are determined for IMD 16. As another example, the clinician may utilize clinician programmer 22 to determine a target value that indicates a positive patient mood state, and, therefore, a desired mood state metric outcome during therapy delivery.

FIG. 11 is a schematic diagram illustrating different examples of sensing module 26 (FIG. 1) and/or sensing module 33 that may be used to monitor a brain signal at two or more locations along a mood circuit and/or one or more secondary indicators of a mood state of patient 14. As indicated above with respect to FIG. 1, signals generated by sensing module 26, which may be implanted or external to patient 14, may be transmitted to IMD 16 or at least one of programmers 22, 24 via wireless signals or a wired connection. In other examples, sensing module 26 may be incorporated into IMD 16, as shown with respect to sensing module 33 in FIG. 2. IMD 16 or programmers 22, 24 may monitor and analyze the physiological signals from sensing module 26 and/or sensing module 33 to control delivery of a therapy based on brain signals monitored at two or more locations along a mood circuit associated with the patient's psychiatric disorder. For example, the IMD 16 or programmers 22, 24 may monitor the brain signals via sensing module 26 and/or sensing module 33, and evaluate the power level within specific frequencies of the brain signals relative to one another to determine the mood state of patient 14 indicated by the monitored brain signals. In some examples, IMD 16 may control delivery of therapy to patient based on the monitored brain signals, e.g., as described previously with respect to FIGS. 5-8.

As previously described, sensing module 26 and/or sensing module 33 may also monitor one or more secondary indicators indicative of patient mood state in addition to monitoring brain signals at two or more locations of the same mood circuit. In some examples, the secondary indicators monitored by sensing module 26 and/or sensing module 33 may include one or more physiological parameters that may be indicative of the mood state of patient 14. Examples of physiological parameters that may be indicative of patient mood state include a patient's heart rate, respiration rate, electrodermal activity, muscular activity, and the like. In some examples, a secondary indicator may include one or more indirect measures of patient mood state, including measures of patient's activity, which may by indicative of the mood state of patient 14. In some examples, user feedback indicating the mood state of a patient, e.g., feedback from patient 14 or a clinician communicated via programmer 22, 24, may be used as a secondary indicator of patient mood state.

In some examples, sensing module 26 and/or sensing module 33 may include ECG electrodes, which may be carried by an ECG belt 120. ECG belt 120 incorporates a plurality of electrodes for sensing the electrical activity of the heart of patient 14. In the example shown in FIG. 11, ECG belt 120 is worn by patient 14. The heart rate and, in some examples, ECG morphology of patient 14 may be monitored based on the signal provided by ECG belt 120. Examples of suitable ECG belts for sensing the heart rate of patient 14 are the “M” and “F” heart rate monitor models commercially available from Polar Electro OY of Kempele, Finland. In some examples, instead of ECG belt 120, patient 14 may wear a plurality of ECG electrodes (not shown in FIG. 6) attached, e.g., via adhesive patches, at various locations on the chest of patient 14, as is known in the art. An ECG signal derived from the signals sensed by such an array of electrodes may enable both heart rate and ECG morphology monitoring, as is known in the art. In addition to or instead of ECG belt 120, IMD 16 may sense the patient's heart rate, e.g., using electrodes on a housing of IMD 16, electrodes of leads 20, electrodes coupled to other leads or any combination thereof.

In other examples, sensing module 26 and/or sensing module 33 may include a respiration belt 122 that outputs a signal that varies as a function of respiration of the patient may also be worn by patient 14 to monitor activity to determine whether patient 14 is in a particular mood state. For example, in an anxious mood state, the patient's respiration rate may increase relative to a baseline respiration rate associated with a non-anxious mood state of patient 14. Respiration belt 122 may be a plethysmograpy belt, and the signal output by respiration belt 122 may vary as a function of the changes is the thoracic or abdominal circumference of patient 14 that accompany breathing by patient 14. An example of a suitable respiration belt is the TSD201 Respiratory Effort Transducer commercially available from Biopac Systems, Inc. of Goleta, Calif. Alternatively, respiration belt 122 may incorporate or be replaced by a plurality of electrodes that direct an electrical signal through the thorax of patient 14, and circuitry to sense the impedance of the thorax, which varies as a function of respiration of patient 14, based on the signal. The respiration belt may, for example, be used to generate an impedance cardiograph (ICG), which detects properties of blood flow in the thorax. In some examples, the ECG and respiration belts 120, 122 may be a common belt worn by patient 14.

In some examples, sensing module 26 and/or sensing module 33 may also include electrode 124, which may be a surface electrode or intramuscular electrode. Electrode 124 may be positioned to monitor muscle activity (e.g., EMG), the temperature of the patient's facial skin (e.g., a thermal sensing electrode), or the moisture level of the patient's skin (e.g., via electrodermal activity). Alternatively, electrode 124 may be positioned to monitor the muscle activity, temperature, moisture level or extent of perfusion of other regions of the patient's body, such as an arm, leg or torso. Electrode 124 may be coupled to clinician programmer 22, or another device, which may monitor the signals sensed by electrode 124 and transmit the signals to clinician programmer 22.

In some examples, sensing module 26 and/or sensing module 33 may also include activity monitor 128, shown as part of a wrist band in the example of FIG. 11, which outputs a signal that varies as a function of patient movement. Activity monitor 128 may be located anywhere with respect to patient 14. Activity monitor 128 may include a motion sensing component, such as, e.g., an accelerometer, positioned to sense patient movement throughout the day. If the motion sensing component is worn on the wrist of patient 14 as in the example of FIG. 11, monitor 128 may specifically sense the movement of the hand of patient 14. In this manner, monitor 128 may be used to identify repetitive patient movement, such as, the act of hand washing, which may be indicative of an OCD mood state.

More generally, the motion of patient 14, or lack thereof, sensed by monitor 128 may be used to identify periods of activity and inactivity of patient 14. In this manner, monitor 128 may be used to identify mood states of patient that may be associated with activity and inactivity of patient mood states of patient 14. For example, motion sensed by monitor 128 may be indicative of a manic mood state indicated when a relatively high amount of activity versus inactivity is sensed and/or periods of activity during those periods in which inactivity would be expected, e.g., period in which patient 14 would typically be sleeping. As another example, motion sensed by monitor 128 may be indicative of depressive mood state, such as, e.g., when a relatively high amount of inactivity versus activity is sensed over an extended period of time and/or periods of inactivity which would indicate frequent napping by patient 14.

In some examples, sensing module 26 and/or sensing module 33 may also include sense electrodes 126a, 126b (collectively “electrodes 126”), which may be positioned to monitor one or more electrical signals within brain 12 other than that of the brain signals sensed by sensing module 26 and/or sensing module 33 at two or more locations of the same mood circuit. In general, the brain signals sensed by electrodes 126 may be indicative of the mood state of patient 14. In some examples, electrode 126a may be positioned to monitor the power within the alpha frequency band of the left hemisphere of the brain of patient 14, and electrode 126b may be positioned to monitor the power within the alpha frequency band of the right hemisphere. In some patients, the amount of alpha band power in the left hemisphere of the brain relative to the amount of alpha band power in the right hemisphere of the brain may be indicative of a depressive mood state of patient 14. In particular, a relatively large asymmetry between the alpha band powers in the respective hemisphere may be indicative of a depressive mood state. When the asymmetry between the alpha band power decreases, it may be indicative of the mood state shifting from a depressive mood state to another, e.g., a relatively positive mood state. Based on this relationship, sensing module 26 and/or sensing module 33 may monitor brain signals via electrodes 126 as a secondary indicator of patient mood state.

Each of the types of sensing device 120, 122, 124, 126 and 128 described above may be used along or in combination with each other, as well as in addition to other sensing devices capable of sensing other appropriate secondary indicators indicative of a patient's mood state. These secondary indicators may be used to “double check” a determination of the mood state of patient 14 based on the mood state indicated by first and second brain signals monitored at different locations on the same mood circuit of brain 12 of patient 14, as previously described.

FIG. 12 is a flow diagram illustrating an example technique for comparing the mood state indicated by brain signals monitored at different locations of the same mood circuit within brain 12 of patient 14 to the mood state indicated by one or more secondary indicators. The mood state determinations based on the brain signals sensed at different portions of a mood circuit and based on the secondary indicators may be to control therapy delivery.

As described with respect to FIG. 5, sensing module 26 and/or sensing module 33 may monitor first and second brain signals at first and second locations, respectively, of the same mood circuit (90), and processor 34 of IMD 16 (or another device) may determine the mood state indicated by the first and second brain (132). For example, processor 34 may determine a mood state metric indicative of a relationship between a first frequency band characteristic of the first brain signal and a second frequency band characteristic of the second brain signal. The mood state metric may be associated with a patient mood state, e.g., using the technique described with respect to FIG. 9.

Processor 34 may also determine a secondary indicator of patient mood state (134), e.g., based on other physiological signals sensed by sensing module 26 and/or sensing module 33. For example, processor 34 may associate a low level of patient activity, as indicated by activity monitor 128, with a depressive mood state of patient 14. As another example, processor 34 may determine a pattern in the patient's hand movements via the activity monitor 128, which may indicate an obsessive-compulsive mood state. Other types of secondary mood state determinations are described above. In some examples, the mood state of patient 14 may be indicated by a clinician, e.g., based on an objective evaluation of the patient's mood state. For example, as described above with respect to FIG. 9, the clinician indication may be based on a patient's response to various questions, e.g., those related to Beck Depression Inventory, Hamilton Rating Scale for Depression (HAM-D), the Montgomery-Asberg Depression Rating Scale (MADRS) and/or Yale-Brawn Obsessive Compulsive Scale (Y-BOCS). Additionally or alternatively, in some examples, patient mood state may be indicated by a patient 14, e.g., via clinician programmer 22 or patient programmer 24, based on the patient's subjective assessment of their mood state.

If processor 34 determines that the mood state determined based on the first and second brain signals monitored at different parts of a mood circuit and the mood state determined based on the secondary indicators are inconsistent (136), processor 34 may generate an inconsistency indication (138). The mood state determinations may be inconsistent if they are not the same. As an example, the mood state determinations may be inconsistent if the mood state determined based on the first and second brain signals monitored at different parts of a mood circuit indicates a depressive mood state, and the mood state determined based on the secondary indicators indicates a manic mood state or a positive mood state. As another example, the mood state determinations may be inconsistent if the mood state determined based on the first and second brain signals monitored at different parts of a mood circuit indicates a severely depressive mood state and the mood state determined based on the secondary indicators indicates a moderately depressive mood state.

The inconsistency indication may be a value, flag, or signal that is stored in memory 35 (FIG. 2) of IMD 16 or transmitted to another device (e.g., programmer 22 or 24). In some examples, upon generation of the inconsistency indication, processor 34 of IMD 16 may not modify therapy delivery to patient 14. Thus, if therapy was being delivered to patient according to a first therapy program at the time the inconsistent mood state determinations were made, processor 34 of IMD 16 may continue controlling signal generator 37 to deliver therapy according to the first therapy program.

In this manner, processor 34 may control signal generator 37 (FIG. 2) to withhold stimulation in situations in which the patient mood state indicated by the first and second brain signals sensed within a common mood circuit of brain 12 may be inconsistent with the actual mood state of patient 14. Such inconsistency may result for a variety of reasons. In some examples, the activity within the mood circuit of a brain may change over time, which may cause the mood state and mood state metric associations to become inaccurate over time. In some cases, brain 12 may adapt the activity within the monitored regions of the mood circuit such in a manner that induces IMD 16 to deliver therapy for a mood disorder, even though the actual mood state of a patient does not call for the delivery of therapy. That is, due to plasticity of brain 12, brain 12 may drive patient 14 into a manic or euphoric state as characterized by brain signals. In this way, brain 12 may manipulate IMD 16 to deliver therapy when therapy may not be necessary.

In some examples, processor 34 may generate an alert when the inconsistency determination is generated (138). The alert may notify a patient and/or clinician, e.g., via programmers 22, 24, that processor 34 has identified an inconsistency between the mood state determinations based on the first and second brain signals and the mood state determination based on one or more second indicators. Upon receiving the alert, the patient 14 may seek clinician attention, and/or a clinician may evaluate the accuracy of the target values associated with the patient mood states and stored in memory 35 of IMD 16. In some cases, one or more target values may be redefined, e.g., using the technique described with respect to FIG. 9, to better reflect activity of brain 12 within a mood circuit during the particular mood state of patient 14.

In some examples, in addition to generating the alert as described above, processor 34 may also deliver therapy to patient 14 even if processor 34 determines that the mood state indicated by the first and second brain signals was inconsistent with the mood state determination based on the secondary indicators. This may be useful because, depending on the severity of the patient's psychiatric disorder, withholding therapy delivery to patient 14 may be undesirable if the mood state determination based on the first and second brain signals was correct. However, because an alert is also generated by processor 34, a clinician may still be alerted to the potential issue, and appropriate action may be undertaken.

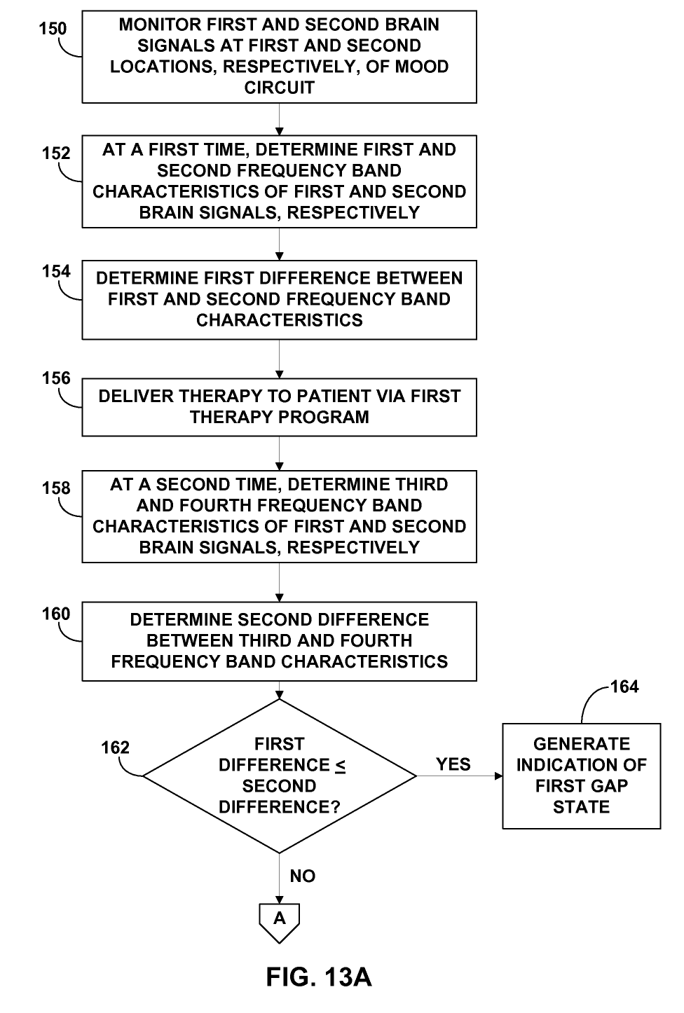

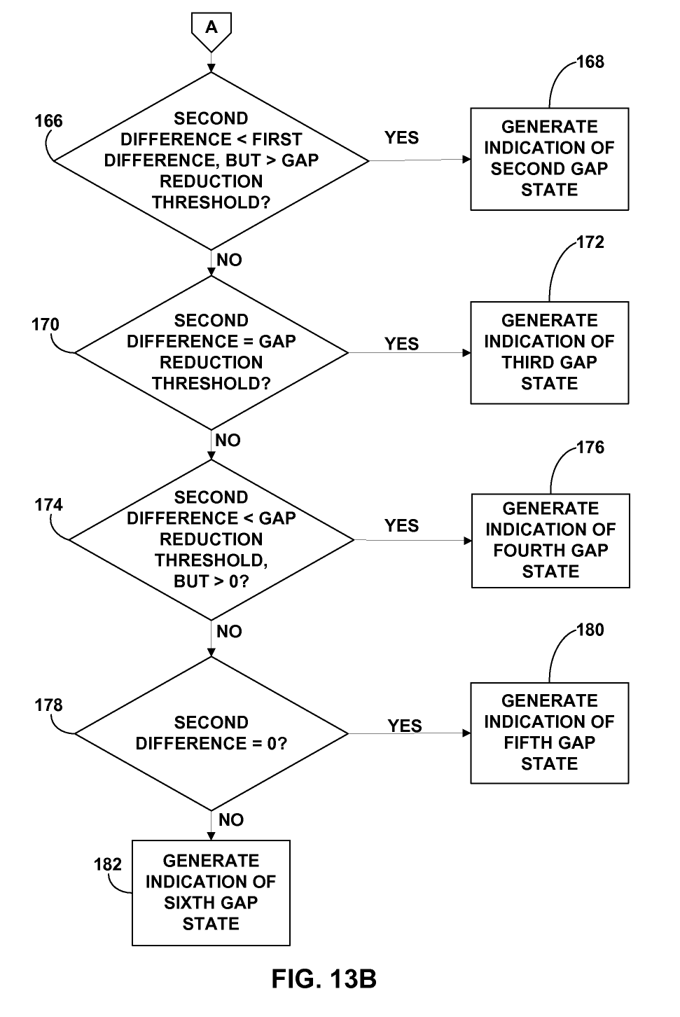

As previously indicated, brain signals sensed within a mood circuit of brain 12 may be useful for selecting one or more therapy parameter values that provide efficacious therapy to patient 14 in managing a psychiatric disorder. Selecting one or more therapy parameter values may involve evaluating one or more therapy programs during a trial stage in which therapy parameter values that provide efficacious therapy to patient 14 are selected by a clinician or automatically selected by IMD 16 or one or both programmers 22, 24. FIGS. 13A and 13B are flow diagrams illustrating an example technique for evaluating one or more therapy programs based on brain activity within a mood circuit of brain 12 that is related to the psychiatric disorder for which IMD 16 provides therapy to control.

Processor 34 of IMD 16 may monitor first and second brain signals and respective locations of a mood circuit via sensing module 26 and/or sensing module 33 (150). As previously described, a mood circuit may generally refer to regions of a brain functionality related to one another via neurological pathways in a manner that causes activity within the respective regions of a common brain circuit to be influenced at least in part based on the mood state of a patient. At a first time, processor 34 may determine first and second frequency band characteristics of the first and second brain signals, respectively (152). The first and second frequency band characteristics may comprise the power level within a particular frequency of the first and second brain signals, respectively. The power levels may be, for example, the average power-in-a-band signals over a period of time, e.g., about one minute or less, although other time periods are contemplated. The first and second frequency band characteristics may or may not be determined within the same frequency bands.

Processor 34 may also determine a mood state metric based on the first and second frequency band characteristics. In the example shown in FIG. 13A, processor 34 determines a first difference between the first and second frequency band characteristics (154). For example, processor 34 may determine the first difference in a first power level of the first brain signal within a frequency band of interest and a second power level of the second brain signal within the frequency band of interest. The first difference between the first and second frequency band characteristics may provide a baseline condition for patient 14, e.g., a condition in which patient 14 is afflicted with a negative mood state and/or prior to delivery of any therapy to patient 14. In general, the baseline condition may represent the patient condition that is undesirable (e.g., because of the presence of a negative mood state), and therapy may be delivered to patient 14 to improve the baseline condition.

In some examples, a particular mood state of patient 14 may be characterized by a difference in the first power level and the second power level. This value may be referred to as a “gap” value because it indicates the difference between the power levels of the first and second brain signals that are sensed within different parts of a mood circuit. In some examples, a negative mood state (e.g., a depressive, anxious or manic mood state) may be characterized by a difference between the first power level and the second power level that exceeds a threshold value. Thus, in some examples, it may be desirable to minimize any difference between the power levels of brain signals sensed within different parts of a mood circuit via therapy delivery by IMD 16. Accordingly, one goal of therapy delivery by IMD 16 may be to achieve a first power level of the first brain signal sensed within a first part of a mood circuit that is within a threshold range of a second power level of the second brain signal that is sensed within a second part of the mood circuit that is different than the first part. In some cases, processor 34 may determine therapy delivery to patient 14 efficacious if the first and second power levels are substantially equal, e.g., such that there is no power-in-a-band asymmetry between brain signals sensed in the different parts of the mood circuit.

After determining the first difference between the first and second frequency band characteristics (154), processor 34 of IMD 16 may control signal generator 37 (FIG. 2) to generate and deliver therapy to patient 14 according to a first therapy program (156). The first therapy program may define values for a first set of stimulation parameters. At a second time after the first time, e.g., after signal generator 37 delivers therapy to patient (156) for a sufficient period of time to enable the therapy to modulate the brain activity, e.g., to modulate the patient's mood state, processor 34 may determine third and fourth frequency band characteristics of the first and second brain signals, respectively, which are sensed within different parts of a mood circuit (158). In some examples, the period of time in which signal generator 37 may deliver therapy to patient 14 prior to the determination of the third and fourth frequency band characteristics may be about 30 seconds to about five minutes or more, although other time periods are contemplated.

In some examples, processor 34 may determine the third and fourth frequency band characteristics of the first and second brain signals while signal generator 37 delivers therapy to patient 14. In other examples, processor 34 may suspend therapy delivery by signal generator 37 prior to determining the third and fourth frequency band characteristics. Processor 34 may determine a second difference between the third and fourth frequency band characteristics (160). As with the first difference between the first and second frequency band characteristics, the second difference may comprise a difference between a power level of the first brain signal and a power level of the second brain signal, where the first and second brain signals are monitored after the therapy delivery according to the first therapy program is initiated.

Processor 34 may determine whether therapy delivery according to the therapy program was successful in changing the patient's mood state by determining whether the first difference substantially equals the second difference (which may be determined in view of appropriate tolerances) or is less than the second difference (162). If the first difference equals the second difference, processor 34 may determine that therapy delivery to patient 14 according to the first therapy program did not change the patient's mood state because the first and second brain signals exhibited similar frequency band characteristics after the therapy was delivered. If the first difference is less than the second difference, processor 34 may determine that the therapy delivered to patient 14 according to the first therapy program changed the mood state of patient in a manner that caused an increase in the difference between the power levels of the first and second signals rather than the desired decrease. In either case, processor 34 may generate an indication of a first gap state (164). The indication of the first gap state, as well as the other indications described herein, may be, for example, a value, flag, or signal that is stored in memory 35 (FIG. 2) of IMD 16 or a memory of another device (e.g., one or both programmers 22, 24) and associated with the therapy program. A clinician may later retrieve the stored indicator and therapy program to evaluate the therapy program.

If the first difference is not less than or equal the second difference, processor 34 may determine whether the second difference is less than the first difference but also greater than a gap reduction threshold (166). The gap reduction threshold may correspond to the threshold difference between the power levels of the first and second brain signals that indicates a positive patient mood state, or at least an improved patient mood state. If the second difference is less than the first difference but also greater than the gap reduction threshold, the first and second brain signals may indicate the power levels in the first and second brain signals are converging in response to therapy delivery according to the first therapy program, but not to an extent that may be considered a positive and/or improved patient mood state. As previously indicated, this may indicate that therapy delivery to patient 14 was successful in modifying the patient's mood state in examples in which it is desirable to minimize the difference between the power levels of the first and second brain signals. If the second difference is less than the first difference and greater than the gap reduction threshold, processor 34 may generate an indication of a second gap state (168), which may be stored in memory 35 of IMD 16 or another device and associated with the therapy program.

If the second difference is not less than the first difference and greater than the gap reduction threshold, processor 34 may determine whether the second difference is substantially equal to a gap reduction threshold (which may be determined in view of appropriate tolerances) (170). As described, the gap reduction threshold may indicate the threshold difference between the power levels of the first and second brain signals that indicates a positive patient mood state, or least an improved patient mood state. If the second difference is substantially equal to a gap reduction threshold, processor 34 may determine that the therapy program provided efficacious therapy to patient 14. The gap reduction threshold may be stored in memory 35 of IMD 16 or another device. If the second difference is substantially equal to a gap reduction threshold, processor 34 may generate an indication of a third gap state (172), which may be stored in memory 35 of IMD 16 or another device and associated with the therapy program.

If the second difference is not substantially equal to a gap reduction threshold, processor 34 may determine whether the second difference is less than the gap reduction threshold (174), but greater than zero. This may indicate that therapy delivery according to the therapy program provided efficacious therapy to patient 14, and was more efficacious than the threshold efficacy level. If the second difference is less than the gap reduction threshold, and greater than zero, processor 34 may generate an indication of a fourth gap state (176), which may be stored in memory 35 of IMD 16 or another device and associated with the therapy program.

If the second difference is not less than the gap reduction threshold and greater than zero, processor 34 may determine whether the second difference is substantially equal to zero (178). This may indicate that the power levels of the first and second brain signals within the respective selected frequency bands are substantially equal, thereby indicating symmetry within the mood circuit. As previously indicated, in some cases, such symmetry between the power levels of brain signals sensed at different portions of a mood circuit may be a marker for a positive patient mood state or at least an improved mood state relative to the baseline condition (discussed above). Processor 34 may determine, if the second difference is substantially equal to zero, that the therapy program defined efficacious therapy parameter values. If the second difference is substantially equal to zero, processor 34 may generate an indication of a fifth gap state (180), which may be stored in memory 35 of IMD 16 or another device and associated with the therapy program. On the other hand, if the second difference is not substantially equal to zero, processor 34 may generate an indication of a sixth gap state (182), which may be stored in memory 35 of IMD 16 or another device and associated with the therapy program.

Processor 34 may evaluate a plurality of therapy programs using the technique shown in FIGS. 13A and 13B. The indications of the first through sixth gap states may be used to communicate information regarding the convergence/divergence of the power levels of the brain signal sensed within a particular mood circuit to a clinician. In this manner, processor 34 may provide guidance to a clinician that is testing different therapy programs (or therapy parameter values) by providing information about how the tested therapy parameter values are affecting the patient's mood state relative to a baseline condition.

Each therapy program maybe associated with an indication of a gap state. Processor 34 or a clinician may determine that the therapy programs associated with a second gap state indication are more efficacious in managing the patient's psychiatric disorder than therapy programs associated with a first gap state indication. Similarly, processor 34 or a clinician may determine that the therapy programs associated with a third gap state indication are more efficacious in managing the patient's psychiatric disorder than therapy programs associated with the second gap state indication. In addition, processor 34 or a clinician may determine that the therapy programs associated with a fifth gap state indication are more efficacious in managing the patient's psychiatric disorder than therapy programs associated with a fourth gap state indication. Processor 34 or a clinician may also determine that the therapy programs associated with a fifth gap state indication are more efficacious in managing the patient's psychiatric disorder than therapy programs associated with a sixth gap state indication.

Therapy programs associated with the sixth gap state indication may be further analyzed by the clinician or processor 34, e.g., to determine whether the therapy programs overcorrected an imbalance between the power levels, such that an imbalance exists, but in a different direction. For example, if the first frequency band characteristic had a greater value than the second frequency band characteristic prior to delivery of therapy according to the therapy program (156), the therapy program may overcorrect the asymmetry between the first and second frequency band characteristics, such that, after therapy delivery according to the first therapy program, the first frequency band characteristic has a lower value than the second frequency band characteristic.

Although the example of FIG. 13 has been described with respect to an example in which it is desirable to minimize the difference between the power levels of the brain signals sensed within different parts of a mood circuit via therapy delivered by IMD 16, examples are not limited to such situations. In some examples, it may be desirable to maximize the difference between the power levels of the brain signals sensed at different parts of a mood circuit via therapy delivered by IMD 16 or at least deliver therapy to achieve a predetermined difference in power levels of two or more brain signals sensed at different parts of a mood circuit. For example, in some cases, a negative mood state may be characterized by a difference between a first power level and second power level that is within a threshold value. In such as example, the technique of FIGS. 13A and 13B may be modified based on the goal of maximizing the difference between the power levels of the brain signals sensed within different parts of the mood circuit via therapy delivery by IMD 16 rather than the goal of minimizing the difference, as previously explained with respect to FIGS. 13A and 13B.

Furthermore, in some examples, processor 34 may be configured to simply determine whether the difference between the power levels in different parts of a mood circuit is converging, diverging or staying approximately the same with respect to a target value or range of value indicative of a positive mood state, and then provide an indication based on the determination.

In each of the examples described herein, processor 34 of IMD 16, processor 40 of patient programmer 24, and/or processor 70 of clinician programmer 22 may store sensed brain signals (e.g., time domain data), as well as frequency band characteristics extracted from the sensed brain signals.

The techniques described in this disclosure, including those attributed to IMD 16, programmer 22, programmer 24, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. While the techniques described herein are primarily described as being performed by processor 34 of IMD 16, processor 40 of patient programmer 24, and/or processor 70 of clinician programmer 22, any one or more parts of the techniques described herein may be implemented by a processor of one of the devices 16, 22, 24, another computing device, alone or in combination with to IMD 16, programmer 22 or programmer 24

In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Various examples of the invention have been described. These and other examples are within the scope of the following claims.

1. A method comprising:
monitoring a first brain signal of a patient at a first location within the brain of the patient;
monitoring a second brain signal at a second location within the brain, wherein the first and second locations are part of a common mood circuit of the brain;
determining a mood state metric indicative of a relationship between a first frequency band characteristic of the first brain signal and a second frequency band characteristic of the second brain signal; and
controlling delivery of therapy to the patient to control a psychiatric disorder based on the mood state metric.

2. The method of claim 1, wherein controlling delivery of therapy comprises selecting one or more therapy parameter values for the therapy based on the mood state metric.

3. The method of claim 2, wherein selecting one or more therapy parameter values for the therapy based on the mood state metric comprises:
delivering the psychiatric disorder therapy to the patient according to a therapy program;
determining the mood state metric after delivering the psychiatric disorder therapy to the patient according to the therapy program;
comparing the mood state metric to a target value; and
storing the therapy program if the mood state metric is within a threshold range of the target value.

4. The method of claim 1, wherein the mood state metric comprises at least one of a ratio of the first frequency band characteristic to the second frequency band characteristic or a difference between the first frequency band characteristic and the second frequency band characteristic.

5. The method of claim 1, wherein the first and second frequency band characteristics comprises a power level within a selected frequency band.

6. The method of claim 1, wherein the first frequency band characteristic comprises a first power level within a first frequency band and the second frequency band characteristic comprises a second power level within a second frequency band that is different than the first frequency band.

7. The method of claim 1, further comprising determining a patient mood state determination based on the mood state metric.

8. The method of claim 7, wherein the patient mood state determination comprises a first patient mood state determination, the method further comprising:
determining a second patient mood state determination based on a secondary indicator that is different than the first and second brain signals;
comparing the first and second mood state determinations; and
generating a consistency determination based on the comparison.

9. The method of claim 8, wherein controlling the delivery of therapy comprises terminating delivery of the therapy if the consistency determination indicates that the first patient mood state determination is inconsistent with the second patient mood state determination.

10. The method of claim 8, wherein controlling the delivery of therapy comprises initiating delivery of the therapy to the patient if the consistency determination indicates that the first patient mood state determination is consistent with the second patient mood state determination.

11. The method of claim 8, wherein the secondary indicator of the patient mood state comprises at least one of patient activity level, cardiac activity, a respiratory rate, electrodermal activity, thermal activity, muscle activity, or user feedback.

12. The method of claim 1, wherein controlling the delivery of therapy to the patient comprises at least one of suspending therapy delivery to the patient or initiating therapy delivery to the patient.

13. The method of claim 1, wherein controlling the delivery of therapy to the patient comprises initiating therapy delivery to the patient, the method further comprising:
comparing the mood state metric to a target value; and
initiating therapy delivery to the patient if the mood state metric is not within a threshold range of the target value.

14. The method of claim 1, wherein controlling the delivery of therapy to the patient comprises:
comparing the mood state metric to a target value; and
adjusting one or more therapy parameter values of the psychiatric disorder therapy if the mood state metric is not within a threshold range of the target value.

15. The method of claim 1, further comprising monitoring a third brain signal at a third location along the mood circuit, wherein determining the mood state metric further comprises determining the mood state metric indicative of the relationship between at least one of the first or second frequency band characteristics and a third frequency band characteristic of the third brain signal.

16. The method of claim 1, wherein the first location and the second location are within different hemispheres of the brain.

17. The method of claim 1, wherein the mood state metric comprises a first mood state metric that indicates a first difference between the first and second frequency band characteristics, and determining the first mood state metric comprises determining the first mood state metric at a first time, and the method further comprises:
determining a second mood state metric indicative of a second difference between a third frequency band characteristic of the first brain signal and a fourth frequency band characteristic of the second brain signal at a second time that occurs prior to the first time;
delivering therapy to the patient according to a therapy program prior to determining the first mood state metric;
determining a gap state based on the first and second mood state metrics; and
associating the gap state with the therapy program in a memory.

18. A medical system comprising:
a therapy module that delivers a psychiatric disorder therapy to a patient;
a sensing module that monitors a first brain signal of a patient at a first location within the brain of the patient and monitors a second brain signal at a second location within the brain, wherein the first and second locations are part of a common mood circuit of the brain; and
a processor that determines a mood state metric indicative of a relationship between a first frequency band characteristic of the first brain signal and a second frequency band characteristic of the second brain signal, and controls the therapy module based on the mood state metric.

19. The medical system of claim 18, further comprising an implantable medical device comprising at least one of the therapy module, sensing module or the processor.

20. The medical system of claim 18, wherein the processor controls the therapy module by at least selecting one or more therapy parameter values with which the therapy module generates the psychiatric disorder therapy based on the mood state metric.

21. The medical system of claim 20, further comprising a memory, wherein the processor selects the one or more therapy parameter values by at least controlling the therapy module to deliver the psychiatric disorder therapy to the patient according to a therapy program, determining the mood state metric after the therapy module initiates delivery of the psychiatric disorder therapy to the patient according to the therapy program, comparing the mood state metric to a target value stored in the memory, and storing the therapy program in the memory if the mood state metric is within a threshold range of the target value.

22. The medical system of claim 18, wherein the mood state metric comprises at least one of a ratio of the first frequency band characteristic to the second frequency band characteristic or a difference between the first frequency band characteristic and the second frequency band characteristic.

23. The medical system of claim 18, wherein the first and second frequency band characteristics comprises a power level within a selected frequency band.

24. The medical system of claim 18, wherein the first frequency band characteristic comprises a first power level within a first frequency band and the second frequency band characteristic comprises a second power level within a second frequency band that is different than the first frequency band.

25. The medical system of claim 18, wherein the processor determines a patient mood state determination based on the mood state metric.

26. The medical system of claim 25, further comprises a memory, wherein the processor determines the patient mood state determination by referencing the memory to determine the patient mood state that is associated with the mood state metric in the memory.

27. The medical system of claim 25, wherein the patient mood state determination comprises a first patient mood state determination, and wherein the processor determines a second patient mood state determination based on a secondary indicator that is different than the first and second brain signals, compares the first and second mood state determinations, and generates a consistency determination based on the comparison.

28. The medical system of claim 27, wherein the secondary indicator comprises at least one of a patient activity level, cardiac activity, a respiratory rate, electrodermal activity, thermal activity, muscle activity or user input indicating a mood state of the patient.

29. The medical system of claim 18, wherein the processor controls the therapy module based on the mood state metric by at least controlling the therapy module to suspend therapy delivery to the patient.

30. The medical system of claim 18, wherein the processor controls the therapy module based on the mood state metric by at least comparing the mood state metric to a target value, and initiating therapy delivery to the patient if the mood state metric is not within a threshold range of the target value.

31. The medical system of claim 18, wherein the processor controls the delivery of therapy by at least comparing the mood state metric to a target value, adjusting one or more therapy parameter values of the psychiatric disorder therapy if the mood state metric is not within a threshold range of the target value, and controlling the therapy module to deliver therapy to the patient according to the adjusted one or more therapy parameter values.

32. The medical system of claim 18, further comprising a memory, wherein the mood state metric comprises a first mood state metric that indicates a first difference between the first and second frequency band characteristics, and the processor determines the first mood state metric at a first time, determines a second mood state metric indicative of a second difference between a third frequency band characteristic of the first brain signal and a fourth frequency band characteristic of the second brain signal at a second time that occurs prior to the first time, controls the therapy module to deliver therapy to the patient according to a therapy program prior to the determination of the first mood state metric, determines a gap state based on the first and second mood state metrics, and associates the gap state with the therapy program in the memory.

33. A medical system comprising:
means for delivering therapy to a patient to control a psychiatric disorder;
means for monitoring a first brain signal of the patient at a first location within the brain of the patient;
means for monitoring a second brain signal at a second location within the brain, wherein the first and second locations are part of a common mood circuit of the brain;
means for determining a mood state metric indicative of a relationship between a first frequency band characteristic of the first brain signal and a second frequency band characteristic of the second brain signal; and
means for controlling the means for delivering therapy based on the mood state metric.

34. The medical system of claim 33, wherein the means for controlling delivery of therapy comprises at least one of means for selecting one or more therapy parameter values for the therapy based on the mood state metric, means for controlling the means for delivering therapy to suspend therapy delivery to the patient, and means for controlling the means for delivering therapy to initiate therapy delivery to the patient

35. The medical system of claim 33, further comprising determining a patient mood state determination based on the mood state metric.

36. The medical system of claim 35, wherein the patient mood state determination comprises a first patient mood state determination, the system further comprising:
means for determining a second patient mood state determination based on a secondary indicator that is different than the first and second brain signals;
means for comparing the first and second mood state determinations; and
means for generating a consistency determination based on the comparison.
